(19) United States
(12) Patent Application Publication
(10) Pub. No.: US 2010/0081666 A1
(43) Pub. Date: Apr. 1, 2010

(54) SRC ACTIVATION FOR DETERMINING CANCER PROGNOSIS AND AS A TARGET FOR CANCER THERAPY

(75) Inventors: Christina M. COUGHLIN, Berwyn, PA (US); Michael E. BURCZYNSKI, Cedar Knolls, NJ (US); Marisa P. DOLLED-FILHART, New Haven, CT (US); Robert PINARD, Andover, MA (US); Donald WALDROM, Fairfield, CT (US); Charles ZACHARCHUK, Westford, MA (US); Frederick IMMERMANN, Suffern, NY (US); Maha KARNOUB, Doylestown, PA (US); Jason CHRISTIANSEN, Glastonbury, CT (US); Mark GUSTAVSON, Niantic, CT (US); Annette MOLINARO, New Haven, CT (US); Alpana Waldron, Fairfield, CT (US)

Correspondence Address: WOMBLE CARLYLE SANDRIDGE & RICE, PLLC;ATTN: PATENT DOCKETING, PO BOX 7037, Atlanta, GA, 30357-0037, US

(73) Assignee: Wyeth, Madison, NJ (US)

(21) Appl. No.: 12/503,019

(22) Filed: Jul. 14, 2009

Related U.S. Application Data
(60) Provisional application No. 61/080,667, filed on Jul. 14, 2008.

(51) Int. Cl. A61K 31/506; C12Q 1/68; C12Q 1/48; C12Q 1/02

(52) U.S. Cl. 514/252.19; 435/6; 435/15; 435/29; 514/253.06; 514/275

(57) ABSTRACT

Methods of cancer diagnosis and prognosis using biomarkers.

### CROSS-REFERENCE TO RELATED APPLICATIONS

Priority is claimed to U.S. Provisional Patent Application No. 61/080,667, filed Jul. 14, 2008, which is incorporated by reference herein in its entirety.

### FIELD OF THE INVENTION

The present invention generally pertains to use of biomarkers of Src activation to identify cancer patient subpopulations with different prognostic outcomes. In particular, the present invention describes significant associations between prognosis and the expression and/or subcellular localization of biomarkers of Src activation, optionally in combination with additional tumor biomarkers. The disclosed methods may further identify cancer patient subpopulations that may benefit from Src inhibition therapy.

### BACKGROUND OF THE INVENTION

Cancer diagnosis and prognosis have historically involved the assessment of clinicopathologic characteristics such as tumor size, nodal involvement, and metastatic spread. The availability of genomic data has provided an additional resource for guiding prognosis and clinical decisions. In particular, understanding signaling pathways that are active in patients that prove resistant to conventional therapy is instrumental to identifying novel combinations of pathway inhibitors to overcome drug resistance in the clinic.

Gene expression data related to oncogenic pathways in breast cancer have recently been shown to have prognostic significance in estimating relapse-free survival and sensitivity to chemotherapy (Acharya et al., JAMA, 2008, 299: 1574-1587). Further, genomic signatures of oncogenic pathway deregulation and predictors of chemotherapy sensitivity were unique for patients with early stage breast cancer, suggesting that targeted treatment strategies may be developed for this patient subpopulation (Anders et al., 2008, PLoS ONE, 3(1): e1373). The application of genetic profiling data remains limited, however, in that no information is obtained regarding the functional molecules encoded by regulated genes.

As a complement to gene expression profiling experiments, other recent studies have investigated the prognostic significance of protein expression and activation, for example, using tissue microarrays for high throughput analysis of intact tissues. In particular, focal adhesion proteins and integrin signaling molecules may be used to predict tumor invasiveness. See Madan et al., Human Pathology, 2006, 37: 9-15. See also Cheang et al., Annu. Rev. Pathol., 2008; 3:67-97; Brennan et al., Cancer Genomics Proteomics, 2007, 4(3):121-134; Zhang et al., Hum. Pathol., 2003, 34(4):362-368; Zhang et al., Mod. Pathol., 2003, 16(1):79-84. For clinical applications of tissue microarrays or similar analysis, an adequate number of patient samples must be represented to enable identification of cancer subtypes and patient subpopulations.

Although many putative biomarkers have been identified using the above-noted approaches, surprisingly few have been successfully developed as assays for clinical prognosis and/or targets for therapy. In view of this continuing need, the present invention provides biomarkers for predicting the prognosis of breast cancer patients, and for identification of cancer patient subpopulations that may benefit from Src inhibition therapy.

### SUMMARY OF THE INVENTION

The present invention provides methods of predicting prognosis of a cancer patient based upon a level of activation of Src signaling. For example, poor prognosis of a cancer patient may be determined by observing or detecting activation of Src signaling in a tumor sample obtained from a patient. Conversely, favorable prognosis of a cancer patient may be determined by observing or detecting suppression of Src signaling in a tumor sample obtained from a patient. The present application further identifies cancer patient populations and subpopulations that may benefit from Src inhibition therapy, i.e., patients having tumors characterized by activated Src signaling, and methods of treating such patients.

In the provided methods, the level of activation of Src signaling, or Src pathway activation, is observed or detected by using any protein or RNA expression analyses known in the art to quantify levels of expression of Src pathway components that serve as surrogate markers of the level of Src pathway activation. Expression levels of a particular Src pathway activation marker are subsequently correlated to a particular prognosis, expected benefit from Src inhibition therapy, or course of treatment. Correlations are provided based upon whether expression levels of a particular Src pathway activation marker are reduced or elevated compared to a control. Correlations are also provided based upon expression level “scoring” and comparison to one or more predetermined cut-points.

In a particular aspect of the invention, a method for predicting favorable prognosis of a cancer patient is performed by (a) observing or detecting reduced expression of HER2, estrogen receptor, and progesterone receptor in a tumor sample obtained from the patient; and (b) observing or detecting reduced expression of paxillin in the tumor sample or detecting an elevated level of phosphorylated Src (pSrc) in the tumor sample. In another aspect of the invention, a method for predicting favorable prognosis of a cancer patient is performed by (a) observing or detecting reduced expression of HER2 in a tumor sample obtained from the patient; and (b) observing or detecting reduced cytoplasmic expression of FAK protein or elevated nuclear expression of FAK protein in the tumor sample.

In another aspect of the invention, a method of predicting poor prognosis of a cancer patient is performed by (a) observing or detecting elevated expression of HER2, estrogen receptor, and progesterone receptor in a tumor sample obtained from the patient; and (b) observing or detecting elevated expression of p130cas in the tumor sample. Predicting poor prognosis of a cancer patient may also be performed by (a) observing or detecting elevated expression of HER2 in a tumor sample obtained from the patient; and (b) observing or detecting elevated expression of paxillin in the tumor sample.

In still another aspect of the invention, a method of predicting the prognosis of a cancer patient is performed by (a) obtaining a biological sample comprising a cancer cell from the cancer patient; (b) subjecting the biological sample to protein or RNA expression analysis; (c) quantifying the protein or RNA expression level of at least one Src pathway activation marker in the biological sample; (d) calculating a score from the protein or RNA expression level of the at least one Src pathway activation marker in the biological sample; and (e) using the score to predict the prognosis of the cancer patient.

The present invention also provides methods of performing an assay useful for predicting prognosis of a cancer patient comprising detecting activation of Src signaling in a tumor sample obtained from the patient, which indicates poor prognosis, or detecting suppression of Src signaling in a tumor sample obtained from the patient, which indicates favorable prognosis. For example, the method can include observing or detecting a level of Src signaling by observing or detecting one or more of expression of p130cas, expression of paxillin, nuclear expression of FAK protein, cytoplasmic expression of FAK protein, and phosphorylation of Src tyrosine kinase. According to the disclosed methods, elevated expression of p130cas, elevated expression of paxillin, reduced nuclear expression of FAK protein, elevated cytoplasmic expression of FAK protein, and elevated phosphorylated Src tyrosine kinase indicate poor prognosis. Conversely, reduced expression of p130cas, reduced expression of paxillin, elevated nuclear expression of FAK protein, reduced cytoplasmic expression of FAK protein, and reduced phosphorylated Src tyrosine kinase, indicate favorable prognosis.

In still other aspects of the invention, a method is provided for identifying cancer patients that may benefit from Src inhibition therapy by observing or detecting one or more of elevated expression of p130cas, elevated expression of paxillin, reduced nuclear expression of FAK protein, elevated cytoplasmic expression of FAK protein, and elevated phosphorylated Src tyrosine kinase, in a tumor sample obtained from the patient. For example, in the case of breast cancer, a method for identifying patients that may benefit from Src inhibition therapy is performed by observing or detecting activated Src signaling and observing or detecting (a) elevated HER2 expression; (b) elevated estrogen receptor expression; (c) elevated progesterone receptor expression; (d) elevated estrogen receptor expression and elevated progesterone receptor expression; or (e) reduced HER2 expression, reduced estrogen expression, and reduced progesterone expression in the tumor sample.

In yet another aspect of the invention, a method of predicting the response of a cancer patient to a Src pathway inhibitor is provided. This method is performed by (a) obtaining a biological sample comprising a cancer cell from the cancer patient; (b) subjecting the biological sample to protein or RNA expression analysis; (c) quantifying the protein or RNA expression level of at least one Src pathway activation marker in the biological sample; (d) calculating a score from the protein or RNA expression level of the at least one Src pathway activation marker in the biological sample; and (e) using the score to predict the response of the cancer patient to the Src pathway inhibitor.

Also provided herein are methods for treating cancer patients, in particular patients having tumors characterized by activated Src signaling, by administering a Src pathway inhibitor, either alone or in combination with one or more additional anti-cancer agents. In one aspect, a method of treating cancer in a cancer patient is performed by (a) obtaining a biological sample comprising a cancer cell from the cancer patient; (b) subjecting the biological sample to protein or RNA expression analysis; (c) quantifying the protein or RNA expression level of at least one Src pathway activation marker in the biological sample; (d) calculating a score from the protein or RNA expression level of the at least one Src pathway activation marker in the biological sample; and (e) administering a Src pathway inhibitor to the cancer patient if the score is greater than or equal to at least one predetermined value.

Having identified patients that will potentially benefit from Src inhibition therapy, treatment of such patients comprising a Src inhibitor administered as monotherapy or within a combination therapy that employs a Src inhibitor as one of multiple therapeutic agents is expected to elicit synergistic therapeutic effects.

### BRIEF DESCRIPTION OF THE DRAWINGS

FIGS. 1A-1D show cluster analysis of phosphorylated Src (pSrc) expression in tumor samples as described in Example 2. FIG. 1A shows cluster number patient distribution for phosphorylated Src expression in tumor samples following two-step unsupervised cluster analysis based on AQUA® score (log2 transformed). TwoStep Cluster Number 1, cluster size=91; TwoStep Cluster Number 2, cluster size=135; TwoStep Cluster Number 3, cluster size=161; TwoStep Cluster Number 4, cluster size=155. FIG. 1B shows AQUA® score cluster distribution for phosphorylated Src expression in tumor samples (simultaneous 95% confidence interval for means; mean=7.46). FIG. 1C shows numerical cluster centroid data for phosphorylated Src expression in tumor samples, shown in FIG. 1B. Based on the distribution around the mean, the two highest clusters and the two lowest clusters were combined to form “High” and “Low” clusters respectively. FIG. 1D shows Kaplan Meier analysis of survival functions in high phosphorylated Src expressing clusters versus low phosphorylated Src expressing clusters (P=0.027).

FIGS. 2A-2C show cluster analysis of p130cas expression in tumor regions of samples as described in Example 3. FIG. 2A shows cluster number patient distribution for p130cas expression following two-step unsupervised cluster analysis based on AQUA® scores (log2 transformed). TwoStep Cluster Number 1, cluster size=237; TwoStep Cluster Number 2, cluster size=287. FIG. 2B shows AQUA® score cluster distribution for p130cas expression (simultaneous 95% confidence interval for means; mean=6.15). FIG. 2C shows Kaplan Meier analysis of survival functions for the two highest p130cas expressing clusters versus lowest p130cas expressing clusters (P=0.245).

FIGS. 3A-3C show cluster analysis of p130cas expression in tumor cytoplasm samples as described in Example 3. FIG. 3A shows cluster number patient distribution for p130cas cytoplasmic expression following two-step unsupervised cluster analysis based on AQUA® scores (log2 transformed). TwoStep Cluster Number 1, cluster size=76; TwoStep Cluster Number 2, cluster size=156; TwoStep Cluster Number 3, cluster size=182; TwoStep Cluster Number 4, cluster size=111. FIG. 3B shows AQUA® score cluster distribution for p130cas cytoplasmic expression (simultaneous 95% confidence interval for means; mean=6.15). FIG. 3C shows Kaplan Meier analysis of survival functions for the two highest p130cas cytoplasmic expressing clusters versus the two lowest p130cas expressing clusters (P=0.066).

FIGS. 4A-4C show cluster analysis of paxillin expression in tumor samples as described in Example 4. FIG. 4A shows cluster number patient distribution for paxillin expression following two-step unsupervised cluster analysis based on AQUA® scores (log2 transformed). TwoStep Cluster Number 1, cluster size=216; TwoStep Cluster Number 2, cluster size=287. FIG. 4B shows AQUA® score cluster distribution for paxillin expression (simultaneous 95% confidence interval for means; mean=7.31). FIG. 4C shows Kaplan Meier analysis of survival functions for high paxillin expressing clusters versus low paxillin expressing clusters (P=0.104).

FIGS. 5A-5C show cluster analysis of FAK expression in tumor cell nuclei as described in Example 5. FIG. 5A shows cluster number patient distribution for FAK nuclear expression following two-step unsupervised cluster analysis based on AQUA® scores (log2 transformed). TwoStep Cluster Number 1, cluster size=63; TwoStep Cluster Number 2, cluster size=258; TwoStep Cluster Number 3, cluster size=184. FIG. 5B shows AQUA® score cluster distribution for FAK nuclear expression (simultaneous 95% confidence interval for means; mean=3.44). FIG. 5C shows Kaplan Meier analysis of survival functions for high nuclear FAK expressing clusters versus low FAK nuclear expressing clusters (P=0.034).

FIGS. 6A-6B show cluster analysis of HER2 expression in tumor samples (log2 transformed) and Kaplan Meier Analysis of survival functions for the same expressing clusters (FIG. 6B; simultaneous 95% confidence interval for means; mean=6.18), as described in Example 6.

FIGS. 7A-7B show cluster analysis of ER nuclear expression in tumor samples (FIG. 7A; log2 transformed) and Kaplan Meier Analysis of survival functions for the same expressing clusters (FIG. 7B; simultaneous 95% confidence interval for means; mean=5.92), as described in Example 7.

FIGS. 8A-8B show cluster analysis of PR nuclear expression in tumor samples (FIG. 8A; log2 transformed) and Kaplan Meier Analysis of survival functions for the same expressing clusters (FIG. 8B; simultaneous 95% confidence interval for means; mean=6.14), as described in Example 8.

FIG. 9 depicts Multiple Correspondence Analysis (MCA) demonstrating relationship between biomarker expression cluster groups for the indicated biomarkers and patient prognosis as described in Example 9. ER, nuclear expression; FAK, cytoplasmic expression; HER2, cytoplasmic expression; p130cas, cytoplasmic expression; paxillin, cytoplasmic expression; phosphorylated Src (pSrc), cytoplasmic expression; PR, nuclear expression.

FIG. 10 shows the correlation between the level of expression of biomarkers of Src activation and levels of HER2 expression in patients.

FIG. 11 shows the correlation between the level of expression of biomarkers of Src activation and levels of HER2, estrogen receptor (ER), and progesterone receptor (ER) expression in patients.

FIG. 12 is a histogram of binned phosphorylated Src (pSrc) AQUA® scores (log2 transformed) from an ER and/or PR positive, HER2 negative patient subpopulation showing the pSrc cut-point that was determined via an unsupervised two-step cluster analysis. SRC-OFF patients are indicated by light-colored bars and SRC-ON patients are indicated by dark-colored bars. The height of each bar, provided above each bar, represents the number of records in that particular bin.

FIG. 13 shows a CART-based assessment of Src pathway markers used to determine the status of Src pathway activation.

FIG. 14 is a histogram of binned phosphorylated Src (pSrc) AQUA® scores (log2 transformed) from an ER and/or PR positive, HER2 negative patient subpopulation. The highest and lowest quartiles are indicated by dotted lines.

FIG. 15 shows a different CART-based assessment of Src pathway markers used to determine the status of Src pathway activation.

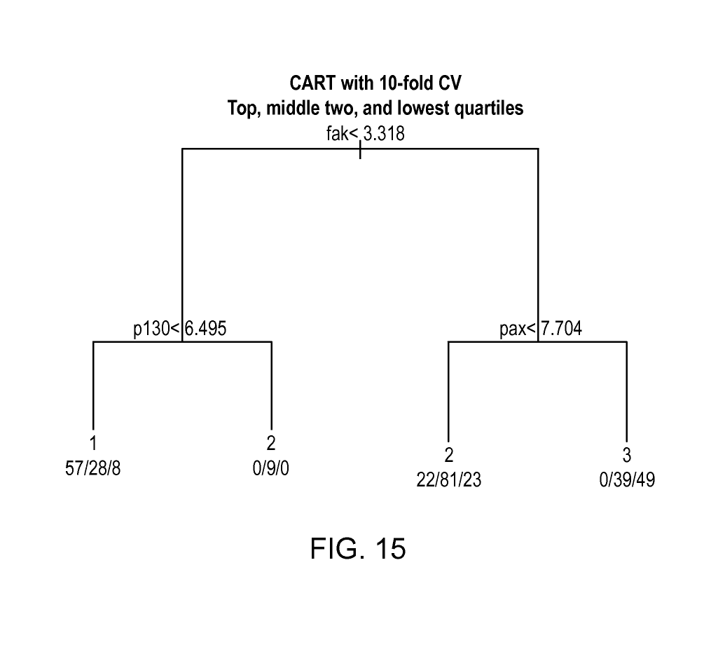

### DETAILED DESCRIPTION OF THE INVENTION

The present invention provides methods for diagnosis of cancer in a patient using biomarkers of Src pathway activation/Src signaling, optionally in combination with one or more additional biomarkers. The particular combination of biomarkers further enables a determination of good or poor prognosis and identification of patients that may benefit from Src pathway inhibition therapy.

### I. Biomarkers of Src Pathway Activation

Non-receptor tyrosine kinases including FAK (Focal Adhesion Kinase) and Src (cellular Src) form a dual kinase complex that is activated in many tumor cells. In both normal and cancerous cells, integrin-regulated pathways exist to recruit and activate FAK or Src. Activated FAK-Src functions to promote cell motility, cell cycle progression, and cell survival, which in cancer cells, leads to tumor growth and/or cancer progression and metastasis.

The present invention provides biomarkers for assessing activation of the Src signaling pathway, which are useful in cancer diagnosis and prognosis. As described further herein below, the Src activation biomarkers can be used alone or in combination with additional cancer cell markers to further refine diagnosis and/or prognosis. In the methods disclosed herein that specify determining activation of the Src pathway, such determination encompasses detecting altered levels of protein or RNA components of Src signaling, detecting altered expression or activity of upstream components of Src signaling, detecting elevated expression or activity of downstream components of Src signaling, detecting protein activation of Src signaling pathway components, or detecting pheontypic changes that indicate Src signaling (e.g., VEGF-associated tumor angiogenesis, protease-associated tumor metastasis, cell spreading, locomotion, survival, anchorage-dependent growth, resistance to apoptosis, etc.).

As used herein, the descriptions “Src signaling” and “Src signaling pathway” refer to genes and proteins that function upstream, in concert with, and downstream of the Src tyrosine kinase. Proteins that are “upstream” of the Src protein act upon the Src protein, i.e., Src is a direct or indirect substrate for these proteins, which regulate Src tyrosine kinase activity. Proteins that act in concert with Src are those proteins that may bind to or form part of a heterogeneous complex with Src protein to thereby regulate its activity. Proteins that are “downstream” of the Src protein are acted upon by the Src protein, i.e., they are direct or indirect substrates of Src tyrosine kinase activity.

Activated Src results in activation of Ras, a prototypic member of the low-molecular weight family of protein GTPases which cycles between an inactive GDP-bound state and an active GTP-bound state, which in turn activates Raf and controls downstream cellular events (Boguski et al., Nature, 1993, 366:643-653). Activated Src has also been shown to bypass activation of Ras-GTP complexes to activate Raf in a Ras-independent manner (Stokoe et al., EMBO J., 1997, 16:2384-2396). Activated Raf then phosphorylates and activates Mitogen-Activated Protein Kinase Kinase (MEK) (Dent et al., Science, 1992, 257:1404-1407; Howe et al., Cell, 1992, 71:335-342), which in turn phosphorylates both tyrosines and threonines of the extracellular-signal-regulated protein kinases (ERKs), members of the MAP kinase (MAPK) family. Activated Src also acts independently of the Ras/Raf signaling cascade to activate the nuclear factor Myc, among other proteins and kinases (reviewed in Erpel et al., J. Biol. Chem., 1995, 271:16807-16812).

For example, oncogenic Src signaling pathway components include kinases such as Src, FAK (Focal Adhesion Kinase), CSK, RAF1, FYN, MEK1, MEK2, ERK1, ERK2, MAPK, JNK, and ROCK1; adaptor proteins such as p130cas, paxillin, and SHC; phosphatases such as MLCP and BCR (breakpoint cluster region)/ABL; growth factor regulatory proteins such as growth factor receptor-bound protein 2 (GRB2); signal transduction proteins such as Signal Transducer and Activator of Transcription molecules (e.g., STAT3, STATS, STATE, and phosphorylated versions thereof) and Rap guanine nucleotide exchange factor (GEF) 1 (C3G), ras homolog gene family (RHO) proteins; transcription factors such as C-JUN and MYC; transmembrane proteins such as caveolin and integrins, including integrin-β and integrin-α; structural proteins such as actin, F-actin, talin, Calpain, α-actinin, myosin, tensin, vinculin, zyxin, and actopaxin; regulators of actin organization, such as COOL/PIX and PKL/GIT; and other proteins found at focal adhesions. The oncogenic activity of the Src pathway is believed to center around the ability of Src to alter cellular structure (e.g., the actin cytoskeleton and the adhesion networks) that control cellular migration via RhoA-ROCK signaling components, the activities of FAK and paxillin that support cellular migration and invasion, and transduction signals that activate and potentiate cellular proliferation and survival (e.g., via STAT transcription factor activation, direct phosphorylation of integrin, and Ras activity).

The Src signaling pathway includes upstream regulatory proteins that function to activate or enhance Src tyrosine kinase activity and also proteins that deactivate or suppress Src tyrosine kinase activity. Oncogenic Src is thought to potentiate signaling via cell surface receptor tyrosine kinases known to play a role in oncogenesis such as the ErbB family (including Her2 and EGFR) as well as insulin like growth receptor (IGF-1R). Thus, determining activation of Src signaling can include assessing the activity of upstream components that promote Src signaling as well as assessing the activity of components that result in Src disinhibition.

I.A. Altered Expression Levels of Src Signaling Pathway Components

Detecting altered levels of Src signaling pathway components in a tumor sample (i.e., a sample containing cancer cells) may be accomplished by detecting altered RNA or protein levels of a particular component when compared to a control level. Relevant controls include RNA or protein levels in a non-cancer cell of the same type as a cancer cell, or RNA or protein levels in a cancer cell prior to receiving an indicated treatment. Such control levels may be measured concomitantly with detecting levels of Src signaling pathway components in a cancer cell or test cell, before or after detecting levels of Src signaling pathway components in a cancer cell or test cell, or may constitute known levels in a control cell such that repeated determination is not required.

Expression analysis of Src signaling pathway components may be determined by detecting protein or RNA using techniques well known to one skilled in the art. The invention may be successfully performed using any suitable detection technique that generates a quantifiable result.

For example, protein expression levels may be determined by immunoassays, Western Blot analysis, or two-dimensional gel electrophoresis. Representative immunoassays include immunohistochemistry (including tissue microarray formats), fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). Protein levels may also be detected based upon detection of protein/protein interactions, including protein/antibody interactions using techniques such as Fluorescence Correlation Spectroscopy, Surface-Enhanced Laser Desorption/Ionization Time-Of-flight Spectroscopy, and BIACORE® technology.

RNA expression levels may be determined using techniques such as reverse-transcriptase polymerase chain reaction (RT-PCR), quantitative reverse-transcriptase polymerase chain reaction (QRT-PCR), TAQMAN® real-time-PCR fluorogenic assay, serial analysis of gene expression (SAGE) (see e.g., Velculescu et al., Cell, 1997, 88, 243-251; Zhang et al., Science, 1997, 276, 1268-1272, and Velculescu et al., Nat. Genet., 1999, 23, 387-388), microarray hybridization, Northern Blot analysis, and in situ hybridization.

According to the present invention, levels of expressed protein or RNA of a Src signaling marker in a tumor sample are quantified for comparison with control levels. For in situ analysis, the AQUA® pathology system may be used. In brief, monochromatic, high-resolution (1,024×1,024 pixel; 0.5 μm) images are obtained of each histological sample. Cellular or subcellular (e.g., nuclei, cytoplasm, etc.) areas of interest are identified by creating a mask (e.g., a tumor mask), and the signal within the mask is then used to identify the cellular or subcellular area of interest. AQUA® scores are measured as the intensity of expressed protein within the area of interest and are typically normalized to the mask. AQUA® scores for duplicate tissue cores can be averaged to obtain a mean AQUA® score for each sample.

I.B. Activation of Src Signaling Pathway Components

Detecting activation or suppression of Src signaling may also be performed by detecting an elevated or reduced level, respectively, of a Src signaling protein in an activated state. For example, a Src signaling pathway protein may become activated by association with other molecules to thereby form an activated complex, by disassociation from a complex to thereby become activated, by post-translational changes that influence protein activity (e.g., changes in phosphorylation, oxidation, etc.), by changes in protein conformation or solubility, etc.

Thus, detecting activation of Src signaling can comprise detecting formation of a Src/FAK complex as described herein above. Both Src and FAK are phosphorylated when activated, and therefore, detecting the phosphorylated forms of these molecules may also be used as biomarkers of Src activation. Specifically, FAK is autophosphorylated, which renders the tyrosine residue at position 397 accessible to the SH2 domain of Src. The kinase activity of Src is thereby stimulated, and reverse phosphorylation of FAK occurs at four tyrosine residues in the activation loop of the FAK kinase. This in turn, induces maximum activity of FAK by creating binding sites for downstream signaling components.

The FAK-Src complex then binds to and can phosphorylate various adaptor proteins such as p130Cas and paxillin. Paxillin is tyrosine-phosphorylated by FAK and Src upon integrin engagement or growth factor stimulation, creating binding sites for the adapter protein Crk. p130Cas (Crk-associated substrate) is also tyrosine-phosphorylated protein in response to Src activation, and when phosphorylated, then binds downstream effector molecules, including Crk and C3G. Accordingly, these protein interactions and phosphorylated p130Cas and paxillin proteins are also biomarkers of activated Src signaling.

Techniques for detection of an activated protein state may be used as appropriate for the modification indicative of the activated protein state. Any of the above-noted immunoassays may also be used to detect a protein in an activated state, wherein the modification generates a new antigenic moiety. For example, detection of activated Src, FAK, paxillin, or p130Cas may be accomplished using an antibody that specifically binds to the phosphorylated versions of these proteins. Representative methods for detecting phosphorylated Src are described in Example 2. Techniques for measuring interactions between one or more proteins, as occurs in complex formation, include electrophorectic assays, competitive inhibition assays, Fluorescence Correlation Spectroscopy, Surface-Enhanced Laser Desorption/Ionization Time-Of-flight Spectroscopy, and BIACORE® technology. Techniques for measuring protein conformation include solubility assays, electrophorectic assays, epitope protection assays, kinetic assays (e.g., Kerby et al., Biotechnology Progress, 2006, 22(5):1416-1425), site-specific proteolysis assays, and immunoassays using antibodies that specifically bind an activated protein conformation. One skilled in the art is readily able to select a technique that may be used to detect an activated protein state in accordance with the diagnostic and prognostic methods of the present invention.

I.C. Localization of Src Signaling Pathway Components

Activation of Src signaling may also be detected by assessing localization of Src pathway components. For example, localization of FAK via its C-terminal Focal Adhesion Targeting domain to focal complexes/adhesions (sites of integrin receptor clustering) is a prerequisite for FAK activation. This localization is detected as a reduction in the level of nuclear FAK protein and/or increase in the level of cytoplasmic FAK protein. Following integrin receptor activation, FAK recruits paxillin and p130Cas to focal adhesions. Accordingly, localization of FAK, paxillin, and p130Cas at focal adhesions may be useful biomarkers of activation of Src signaling.

Techniques that may be used for detecting localization of Src signaling components are known in the art, and include numerous immunoassays for detecting levels of protein expression or levels of activated proteins, as described herein above. In some aspects of the invention, subcellular localization of Src signaling components is assessed in combination, either sequentially or contemporaneously, with levels of expression of Src signaling components. For example, reduced expression of nuclear FAK protein is a biomarker for activated Src signaling, such that tumor samples with high nuclear FAK protein are correlated with a survival advantage and tumor samples with low nuclear FAK protein are correlated with a survival disadvantage. See Example 5. Conversely, elevated expression of cytoplasmic FAK protein is a biomarker for suppression of Src signaling, such that tumor samples with elevated cytoplasmic FAK protein are correlated with a survival advantage and tumor samples with reduced cytoplasmic FAK protein are correlated with a survival disadvantage. See Example 10. For example, for patients with tumor samples expressing low levels of HER2, poor prognosis is associated with increased cytoplasmic FAK protein and favorable prognosis is associated with decreased cytoplasmic FAK protein. See Examples 5 and 10.

As for measuring levels of expression of Src signaling pathway components, levels of protein localized to a particular subcellular compartment or specialization (e.g., a focal adhesion) are quantified for comparison to control levels. Thus, in one aspect of the invention provides a method of determining a prognosis of a patient by assessing the relative levels of one or more Src signaling biomarkers in subcellular compartments or specializations of a tissue sample by (a) incubating the tissue sample with a stain that specifically labels a first marker that defines a first subcellular compartment or specialization, a second stain that specifically labels a second marker that defines a second subcellular compartment or specialization, and a third stain that specifically labels a Src signaling biomarker; (b) obtaining a high resolution image of each of the first, second, and third stains in the tissue sample; (c) assigning each pixel of the image to the first or second subcellular compartments or specializations based upon the first and second stain intensities, respectively; (d) measuring the intensity of the third stain in each of the pixels of the image; (e) determining a staining score indicative of the concentration of the biomarker in the first and second subcellular compartments or specializations; and (f) predicting prognosis of the cancer patient based upon the level of the Src signaling biomarker in the first or second subcellular compartment or specializations.

For example, using the AQUA® pathology system, nuclear protein may be quantified as follows. The tissue may be “masked” using cytokeratin in one channel to identify the area of tumor and to remove the stromal and other non-tumor material from analysis. Then an image is taken using DAPI to define a nuclear compartment. The pixels within the mask and within the DAPI-defined compartment are defined as tumor nuclei pixels. The intensity of expression of the protein is measured using a third channel. The intensity of protein expression in the defined subset of pixels divided by the number of pixels (to normalize the area from sample to sample) gives an AQUA® score. This score is directly proportional to the number of molecules of the protein per unit area of tumor nuclei. This technique, including details of out-of-focus light subtraction imaging methods, is described in detail in Camp et al., Nat Med., 2002, 8:1323-1327. See also U.S. Pat. No. 7,219,016. The disclosures of the foregoing references are incorporated herein be reference in their entireties, particularly with respect to the disclosure of techniques for determining AQUA® scores in cellular samples, which may also be used in the methods of the present invention.

Localization of Src signaling biomarkers within cells may also be determined using subcellular fractionation techniques, as known in the art, when used in conjunction with immunoassay techniques. For some biomarkers (e.g., p130cas), detecting changes in levels of expression yields a similar result whether such levels are measured in whole cells or in a subcellular compartment (e.g., nuclear or cytoplasmic expression). For these biomarkers, detection may be alternatively be performed by assessing expression in tumor cells, tumor cell nuclei, tumor cell cytoplasm, or other subcellular compartment of tumor cells, as convenient.

I.D. Quantification of Activation of Src Signaling

When assessing a level of any one of the above-noted criteria (e.g., a level of RNA or protein of a Src signaling component or other tumor marker, a level of activated Src signaling protein, a level of subcellular localization of Src signaling components), the level is assessed relative to a control level. For example, a relevant control may comprise a sample taken from a tumor-bearing patient and from a same tissue and analogous region on the contralateral side of the patient. As another control, a sample may be taken from a same tissue and analogous region from a similarly situated (age, gender, overall health, etc.) patient who lacks a tumor. In the case of assessment of treatment-dependent response, post-treatment effects may also be ascertained through parallel analysis of a pre-treatment control sample.

When quantifying a level of any of the above-described criteria for defining activation or suppression of Src signaling, a difference when assessed relative to a control level is identified as a difference of at least about two-fold greater or less than a control level, or at least about five-fold greater or less than a control level, or at least about ten-fold greater or less than a control level, at least about twenty-fold greater or less than a control level, at least about fifty-fold greater or less than a control level, or at least about one hundred-fold greater or less than a control level. A difference in the above-noted criteria when assessed relative to a control level may also be observed as a difference of at least 20% compared to a control level, such as at least 30%, or at least 40%, or at least 50%, or at least 60%, or at least 70%, or at least 80%, or at least 90%, or at least 100%, or more.

I.E. Tumor Samples

Types of cancer that are amenable to diagnosis or prognosis using the Src signaling biomarkers of the present invention include primary and metastatic tumors in breast, colon, rectum, lung, oropharynx, hypopharynx, esophagus, stomach, pancreas, liver, gallbladder, bile ducts, small intestine, urinary tract including kidney, bladder and urothelium, female genital tract, cervix, uterus, ovaries, male genital tract, prostate, seminal vesicles, testes, an endocrine gland, thyroid gland, adrenal gland, pituitary gland, skin, bone, soft tissues, blood vessels, brain, nerves, eyes, meninges. Representative cancers include fibrosarcoma, myxosarcoma, liposarcoma, chondrosarcoma, osteogenic sarcoma, chordoma, angiosarcoma, endotheliosarcoma, lymphangiosarcoma, synovioma, lymphangioendotheliosarcoma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Hematological malignancies, such as, leukemias and lymphomas, including indolent, aggressive, low-grade, intermediate-grade, or high-grade leukemia or lymphoma are also amenable to the methods of the invention for predicting prognosis. leukemias and lymphomas, including indolent, aggressive, low-grade, intermediate-grade, or high-grade leukemia or lymphoma. Representative B cell malignancies include Hodgkin's lymphoma, B cell chronic lymphocytic leukemia (B-CLL), lymhoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic; follicular, diffuse large cell; diffuse small cleaved cell; large cell immunoblastic lymphoblastoma; small, non-cleaved: Burkitt's and non-Burkitt's: follicular, predominantly large cell; follicular, predominantly small cleaved cell; and follicular, mixed small cleaved and large cell lymphomas. Representative T cell malignancies include T-cell prolymphocytic leukemia, T-cell large granular lymphocytic leukemia, adult T-cell leukemia/lymphoma, cutaneous T-cell lymphoma, and peripheral T-cell lymphoma. Patients having any of the above-identified tumors or B cell malignancies include relapsed patients, or patients who are refractory to prior therapy.

When performing the disclosed methods of detecting biomarkers of Src signaling, the tumor sample is obtained from a patient using customary biopsy techniques. The term “tumor sample” refers to cell or tissue samples obtained from a solid tumor or hematologic malignancy. In some aspects of the invention, a single cell can be used in the analysis. Optionally, one or more cells from a patient may be cultured in vitro so as to obtain a larger population of cells for analysis.

Prior to detection of biomarkers, the tumor sample may be enriched for a particular cell type, for example, malignant cells as compared to non-malignant cells of the tumor microenvironment. Cell subsets may be enriched and/or isolated using known techniques, including FACS using a fluorochrome conjugated marker-binding reagent, attachment to and disattachment from solid phase, magnetic separation, using antibody-coated magnetic beads, affinity chromatography and panning with antibody attached to a solid matrix, e.g., a plate or other convenient support.

When analyzing tissue samples or cells from individuals, it may be important to prevent any further changes in gene expression after the sample has been removed from the patient. Changes in gene expression levels are known to change rapidly following perturbations, e.g., heat shock or activation with lipopolysaccharide (LPS) or other reagents. In addition, RNA and proteins in tissue and cell samples may quickly become degraded. As known in the art, to minimize such changes, a tumor sample obtained from a patient is frozen as soon as possible following procurement from the patient. Tumor samples may be prepared as formalin-fixed, paraffin embedded tissue blocks, and optionally further prepared as a tissue microarray, for example as described in Konenen et al., Nat. Med., 1987, 4:844-847 and Chung et al., Clin. Cancer Res., 2001, 7(12):4013-4020. See also Example 1. Tumor samples may also be prepared for analysis using a reverse phase protein array, for example as described by Grote et al., Proteomics, 2008, 8(15):3051-3060; Tibes et al., Mol. Cancer. Ther., 2006 5(10):2512-2521; and references cited therein.

### II. Additional Biomarkers for Cancer Diagnosis and Prognosis

Biomarkers for any of the above-noted cancers may be used in combination with the disclosed biomarkers for activation of Src signaling. The additional biomarkers may be expressed in tumor tissue or released from a tumor into the blood or other body fluids. The biomarkers may be expressed in numerous cancer types or may be expressed in a limited number or even single cancer type. In some instances, a biomarker may be indicated or a particular cancer subtype.

Representative tumor markers that may be used in combination with the biomarkers for activated Src signaling described herein include 5T4 (e.g., in tumor tissue of patients with solid tumors of bladder, breast, cervix, endometrium, lung, esophagus, ovary, pancreas, stomach, and testes); AFP (Alpha-feto protein) (e.g., in blood of patients with liver cancer or germ cell cancer of ovaries or testes), B2M (Beta-2 microglobulin) (e.g., in blood of patients with multiple myeloma and lymphoma); BTA (Bladder tumor antigen) (e.g., in urine of patients with bladder cancer); CA 15-3 (Cancer antigen 15-3) (e.g., in blood of patients with breast, lung, and ovarian cancers); CA 19-9 (Cancer antigen 19-9) (e.g., in blood of patients with pancreatic cancer, colorectal cancer, bile duct cancer); CA 72-4 (Cancer antigen 72-4) (e.g., in blood of patients with ovarian cancer); CA-125 (Cancer antigen 125) (e.g., in blood of patients with ovarian cancer); CA 15-3 (Cancer antigen 15-3) (e.g., in blood of patients with breast cancer); calcitonin (e.g., in blood of patients with thyroid medullary carcinoma); CEA (Carcino-embryonic antigen) (e.g., in blood of patients with gastrointestinal tract, lung, breast, thyroid, pancreatic, liver, cervix, ovarian, and bladder cancers); EGFR (Her-1) (e.g., in tumor tissue of patients with solid tumors, such as of the lung (non-small cell), head and neck, colon, pancreas, or breast); estrogen receptors (e.g., in tumor tissue of patients with breast cancer, particularly hormone-dependent breast cancer); hCG (Human chorionic gonadotropin) (e.g., in blood or urine of patients with testicular or trophoblastic cancer); HER2/neu (e.g., in tumor tissue of patients with breast cancer); monoclonal immunoglobulins (e.g., in blood or urine of patients with multiple myeloma or Waldenstrom's macroglobulinemia); NSE (Neuron-specific enolase) (e.g., in blood of patients with neuroblastoma or small cell lung cancer); NMP22 (e.g., in urine of patients with bladder cancer); progesterone receptors (e.g., in tumor tissue of patients with breast cancer, particularly hormone-dependent breast cancer); PSA (Prostate specific antigen) (e.g., in blood of patients with prostate cancer); prostate-specific membrane antigen (PSMA) (e.g., in blood of patients with prostate cancer); prostatic acid phosphatase (PAP) (e.g., in blood of patients with metastatic prostate cancer, myeloma, or lung cancer); S-100 (e.g., in blood of patients with metastatic melanoma); TA-90 (e.g., in blood of patients with metastatic melanoma); and thyroglobulin (e.g., in blood of patients with thyroid cancer).

Additional biomarkers may also include genetic markers that indicate a heightened risk of developing cancer (e.g., the mutations BRCA1 and BRCA2 in the case of breast cancer) and gene expression signature profiles having prognostic significance. For example, gene expression signatures associated with activation of TNF-α, RAS, and CTNNB signaling pathways are associated with poor prognosis. See e.g., Acharya et al., JAMA, 2008 299(13):1574-1585. See also Golub, N. Engl. J. Med., 2001, 344(8):601-602; Bild et al., Nature, 2006, 439(7074):353-357; Golub et al., Science, 1999, 286(5439):531-537; Liu et al., N. Engl. J. Med., 2007, 356(3):217-226; Potti et al., N. Engl. J. Med., 2006, 355(6): 570-580; Scharpf et al., Biotechniques, 2003, Suppl:22-29; Massague, N. Engl. J. Med., 2007, 356(3):294-297; Veer et al., Nature, 2002, 415:530-536; West et al., Proc. Natl. Acad. Sci. USA, 2001, 98:11462-11467; Sorlie et al., Proc. Natl. Acad. Sci. USA, 2001, 98:10869-10874; Shipp et al., Nature Medicine, 2002, 8(1):68-74.

In one aspect of the invention, expression of HER2, estrogen receptor, and/or progesterone receptor define breast cancer subtypes, and the disclosed Src signaling biomarkers may be used for determining prognosis of patients with these cancer subtypes. See e.g., Examples 9 and 10 and the discussion below with respect to prognostic methods.

### III. Diagnostic, Prognostic, Predictive and Therapeutic Methods

The disclosed biomarkers of Src signaling have diagnostic (i.e., indicative of malignant transformation), prognostic, predictive and therapeutic applications. When used in combination with additional biomarkers, gene expression signatures, and/or clinicopathologic indicators, the disclosed Src signaling biomarkers may provide a diagnostic, prognostic, or predictive outcome with greater confidence and/or for patients having cancer subtypes. Performance of the diagnostic, prognostic, and predictive methods additionally identifies patient populations wherein activation of Src signaling correlates with poor prognosis and that may therefore benefit from Src inhibition therapy or expect to derive an enhanced benefit from Src inhibition therapy.

III.A. Cancer Prognosis

The term “prognosis” refers to a prediction of how a patient's disease will progress and/or a measurable prediction of possible recovery or disease recurrence. In some instances, prognosis may consider disease progression and possible recovery in response to a particular treatment or therapeutic regimen, and the disclosed Src signaling biomarkers may also be used to monitor responsiveness to a treatment. Measurable indices of favorable prognosis include improved survival rate, temporal extension of disease-free survival, reduction in mortality rate, reduction in incidence of disease recurrence or relapse, and responsiveness to treatment when compared to control values. Likewise, indices of poor prognosis include reduced survival rate, temporal abbreviation of disease-free survival, increased mortality rate, resistance to treatment, and incidence of disease recurrence or relapse when compared to control values.

For cancer, measurable indices of favorable prognosis include clinical outcomes such as reduction in tumor mass and/or the number of nodules related to a hematologic malignancy, reduction of abnormally large spleen or liver, reduction or disappearance of metastases, reduction of tumor invasiveness, reduction of tumor-associated angiogenesis, reduction of the number of malignant cells, reduced or slowed growth of malignant cells, and depletion of antigen presenting cells such as macrophages or dendritic cells from the tumor microenvironment of a cancer patient when compared to control values. Conversely, measurable indices of poor cancer prognosis include expansion of tumor mass and/or the number of nodules related to a hematologic malignancy, increase spleen or liver size, increased metastases, increased tumor invasiveness, increased tumor vascularization, increased number of malignant cells, stimulated growth of malignant cells, and maintenance or accumulation of antigen presenting cells such as macrophages or dendritic cells in the tumor microenvironment of a cancer patient relative to control values.

When correlating activation of Src signaling with prognosis, a change in any of the above-noted indices of prognosis is assessed relative to a control state, such as a patient's prognosis prior to therapy, or a level observed in a healthy patient (i.e., a patient free of cancer or other disease or disorder characterized by Src activation). For determining poor prognosis, activation of Src signaling in a tumor sample may be compared to a level of Src signaling observed in a tumor sample characterized by inactive or suppressed Src signaling. Conversely, for determining favorable prognosis, suppression of Src signaling in a tumor sample may be compared to a level of Src signaling observed in a tumor sample characterized by activated Src signaling. Where biomarkers other than Src signaling biomarkers define particular patient populations or subpopulations, a control tumor sample is typically taken from a same population or subpopulation as the test tumor sample (e.g., both control tumor sample and test tumor sample are HER2-negative, ER-negative, and PR-negative). One skilled in the art can readily identify appropriate controls for assessing changes in levels of Src activation.

A change in any of the above-noted prognostic indices may be a change of at least about two-fold greater or less than a control level, or at least about at least about five-fold greater or less than a control level, or at least about ten-fold greater or less than a control level, at least about twenty-fold greater or less than a control level, at least about fifty-fold greater or less than a control level, or at least about one hundred-fold greater or less than a control level. A change in the above-noted indices may also be observed as a change of at least 20% compared to a control level, such as at least 30%, or at least 40%, or at least 50%, or at least 60%, or at least 70%, or at least 80%, or at least 90%, or at least 100%, or more. In some cases, a control level of expression may be essentially a lack of expression or an undetectable level of expression. Similarly, a reduction in expression may be a reduction in expression to a level that is essentially a lack of expression or an undetectable level of expression. A change to a state that more closely resembles a control or healthy state, or a state in which Src signaling is suppressed or not activated, indicates a favorable prognosis. Conversely, a change to a state that is less similar to a control or healthy state, or a change to a state in which Src signaling is activated, indicates poor prognosis.

In one aspect of the present invention, a method of predicting poor prognosis of a patient with cancer comprises observing or detecting activation of Src signaling in a tumor sample obtained from the patient, for example, by observing or detecting elevated expression of p130cas, elevated expression of paxillin, reduced nuclear expression of FAK protein, elevated cytoplasmic expression of FAK protein, and elevated phosphorylated Src tyrosine kinase. In another aspect of the invention, a method of predicting favorable prognosis of a patient with cancer comprises observing or detecting suppression of Src signaling in a tumor sample, for example, by observing or detecting reduced expression of p130cas, reduced expression of paxillin, elevated nuclear expression of FAK protein, reduced cytoplasmic expression of FAK protein, and reduced phosphorylated Src tyrosine kinase.

In other aspects of the invention, methods of diagnosis or prognosis are provided for particular breast cancer subtypes, including HER2-positive cancer, estrogen receptor (ER)-positive cancer, progesterone receptor (PR)-positive cancer, and HER2-negative, ER-negative, PR-negative (triple negative) cancer. Specifically, a method of predicting favorable prognosis of a triple negative cancer patient may include the steps of (a) observing or detecting reduced expression of HER2, estrogen receptor, and progesterone receptor in a tumor sample obtained from the patient; and (b) observing or detecting reduced expression of paxillin in the tumor sample. A method of predicting poor prognosis of a cancer patient may include the steps of (a) observing or detecting elevated expression of HER2, estrogen receptor, and progesterone receptor in a tumor sample obtained from the patient; and (b) observing or detecting elevated expression of p130cas in the tumor sample. A method of predicting favorable prognosis of a cancer patient may also include the steps of (a) observing or detecting reduced expression of HER2 in a tumor sample obtained from the patient; and (b) observing or detecting reduced cytoplasmic expression of FAK or elevated nuclear expression of FAK in the tumor sample. As a further alternative, a method of predicting poor prognosis of a cancer patient may include the steps of (a) observing or detecting elevated expression of HER2 in a tumor sample obtained from the patient; and (b) observing or detecting elevated expression of paxillin in the tumor sample. When performing any of the foregoing prognosis predictions, if the HER2, ER, and/or PR profiles are known for a particular patient(s), the methods may be performed by observing or detecting only phosphorylated Src, nuclear or cytoplasmic FAK protein, p130cas, or paxillin as indicated herein.

In still other aspects of the invention are methods of performing an assay useful for predicting prognosis of a cancer patient comprising observing or detecting activation or suppression of Src signaling in a tumor sample obtained from the patient. Such detection methods include any of the methods described herein in the context of methods for predicting favorable or poor prognosis of a cancer patient. For example, the method can include observing or detecting a level of Src signaling by observing or detecting one or more of expression of p130cas, expression of paxillin, nuclear expression of FAK protein, cytoplasmic expression of FAK protein, and phosphorylation of Src tyrosine kinase. The step of detecting activation or suppression of Src signaling may be performed independently from use of the assay results for predicting prognosis of a cancer patient. For example, levels of Src activation in a tumor sample may be determined by performing a detecting step, which results are then useful to another in predicting patient prognosis. Upon receipt and analysis of information pertaining to levels of Src activation in a tumor sample, which information is the result of a detection step previously performed (i.e., observation of levels of Src activation without having performed a step comprising detecting levels of Src activation in a tumor sample), activation of Src signaling may be used to determine a poor prognosis, and suppression of Src signaling may be used to determine a favorable prognosis.

III.B. Predictive and Therapeutic Methods

The identification of patients having cancerous cells characterized by activated Src signaling is useful for selecting patients for Src inhibition therapy because the markers disclosed herein are also predictive in nature. Accordingly, patients exhibiting higher levels of Src signaling (i.e., expression of Src pathway activation markers) would be expected to be more responsive and/or derive an enhanced benefit from a Src pathway inhibitor. Such therapy may include inhibition of any Src signaling pathway component, which results in downregulation of Src signaling (e.g., decreased expression levels of Src pathway activation markers). Representative Src pathway inhibitors include dasatinib, nillotinib, bosutinib (SKI-606), and adenoviral vector expressing the melanoma differentiation-associated gene-7 (Ad-mda7).

For example, as disclosed herein, patients that may benefit from Src inhibition therapy include (1) patients having a HER2-positive breast tumor, which also expresses one or more biomarkers of activated Src signaling; (2) patients having an ER-positive and/or PR-positive breast tumor, and which additionally expresses one or more biomarkers of activated Src signaling; and (3) patients having a HER2-negative, ER-negative, and PR-negative (negative for all three biomarkers) breast tumor, which also expresses one or more biomarkers of activated Src signaling. In one aspect of the invention, biomarkers of activated Src signaling used for patient selection are elevated levels of phosphorylated Src, cytoplasmic FAK protein, p130cas, and/or paxillin proteins.

When treating the patient populations identified herein, measurable therapeutic effects include any of the above-noted effects, i.e., improved survival rate, temporal extension of disease-free survival, reduction in mortality rate, a shift to a more favorable genetic profile, and responsiveness to treatment of a cancer patient when compared to control values.

The present invention further provides methods of treating the afore-mentioned patient groups using a combination of a Src pathway inhibitor and one or more additional anti-cancer agents, wherein the Src pathway inhibitor and the one or more additional anti-cancer agents are administered concurrently or sequentially in any order. The administration of the Src pathway inhibitor and the one or more additional anti-cancer agents preferably elicits a greater therapeutic effect than administration of either alone. For example, a synergistic therapeutic effect may be an effect of at least about two-fold greater than the therapeutic effect elicited by a single agent, or the sum of the therapeutic effects elicited by the single agents of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single agent, or the sum of the therapeutic effects elicited by the single agents of a given combination, or at least 20%, or at least 30%, or at least 40%, or at least 50%, or at least 60%, or at least 70%, or at least 80%, or at least 90%, or at least 100%, or more.

Representative agents useful for combination therapy include cytotoxins, radioisotopes, chemotherapeutic agents, immunomodulatory agents, anti-angiogenic agents, anti-proliferative agents, pro-apoptotic agents, and cytostatic and cytolytic enzymes (e.g., RNAses). A drug may also include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above-noted terms. For example, selected radioisotopes are also cytotoxins.

Patients identified as potentially responsive to Src inhibition therapy may also be treated using a Src pathway inhibitor in combination with a therapeutic antibody or antibody/drug conjugates, including anti-5T4 antibodies, anti-CD19 antibodies, anti-CD20 antibodies (e.g., RITUXAN®, ZEVALIN®, BEXXAR®), anti-CD22 antibodies, anti-CD33 antibodies (e.g., MYLOTARG®), anti-CD33 antibody/drug conjugates, anti-Lewis Y antibodies (e.g., Hu3S193, Mthu3S193, AGmthu3S193), anti-HER-2 antibodies (e.g., HERCEPTIN® (trastuzumab), MDX-210, OMNITARG® (pertuzumab, rhuMAb 2C4)), anti-CD52 antibodies (e.g., CAMPATH®), anti-EGFR antibodies (e.g., ERBITUX® (cetuximab), ABX-EGF (panitumumab)), anti-VEGF antibodies (e.g., AVASTIN® (bevacizumab)), anti-DNA/histone complex antibodies (e.g., ch-TNT-1/b), anti-CEA antibodies (e.g., CEA-Cide, YMB-1003) hLM609, anti-CD47 antibodies (e.g., 6H9), anti-VEGFR2 (or kinase insert domain-containing receptor, KDR) antibodies (e.g., IMC-1C11), anti-Ep-CAM antibodies (e.g., ING-1), anti-FAP antibodies (e.g., sibrotuzumab), anti-DR4 antibodies (e.g., TRAIL-R), anti-progesterone receptor antibodies (e.g., 2C5), anti-CA19.9 antibodies (e.g., GIVAREX®) and anti-fibrin antibodies (e.g., MH-1).

Patients identified as potentially responsive to Src inhibition therapy may also be treated using a Src pathway inhibitor in combination with one or more combinations of cytotoxic agents as part of a treatment regimen. Useful cytotoxic preparations for this purpose include CHOPP (cyclophosphamide, doxorubicin, vincristine, prednisone and procarbazine); CHOP (cyclophosphamide, doxorubicin, vincristine, and prednisone); COP (cyclophosphamide, vincristine, prednisone); CAP-BOP (cyclophosphamide, doxorubicin, procarbazine, bleomycin, vincristine and prednisone); m-BACOD (methotrexate, bleomycin, doxorubicin, cyclophosphamide, vincristine, dexamethasone, and leucovorin; ProMACE-MOPP (prednisone, methotrexate, doxorubicin, cyclophosphamide, etoposide, leukovorin, mechloethamine, vincristine, prednisone and procarbazine); ProMACE-CytaBOM (prednisone, methotrexate, doxorubicin, cyclophosphamide, etoposide, leukovorin, cytarabine, bleomycin and vincristine); MACOP-B (methotrexate, doxorubicin, cyclophosphamide, vincristine, prednisone, bleomycin and leukovorin); MOPP (mechloethamine, vincristine, prednisone and procarbazine); ABVD (adriamycin/doxorubicin, bleomycin, vinblastine and dacarbazine); MOPP (mechloethamine, vincristine, prednisone and procarbazine) alternating with ABV (adriamycin/doxorubicin, bleomycin, vinblastine); MOPP (mechloethamine, vincristine, prednisone and procarbazin) alternating with ABVD (adriamycin/doxorubicin, bleomycin, vinblastine and dacarbazine); ChIVPP (chlorambucil, vinblastine, procarbazine, prednisone); IMVP-16 (ifosfamide, methotrexate, etoposide); MIME (methyl-gag, ifosfamide, methotrexate, etoposide); DHAP (dexamethasone, high-dose cytaribine and cisplatin); ESHAP (etoposide, methylpredisolone, HD cytarabine, and cisplatin); CEPP(B) (cyclophosphamide, etoposide, procarbazine, prednisone and bleomycin); CAMP (lomustine, mitoxantrone, cytarabine and prednisone); and CVP-1 (cyclophosphamide, vincristine and prednisone); DHAP (cisplatin, high-dose cytarabine and dexamethasone); CAP (cyclophosphamide, doxorubicin, cisplatin); PV (cisplatin, vinblastine or vindesine); CE (carboplatin, etoposide); EP (etoposide, cisplatin); MVP (mitomycin, vinblastine or vindesine, cisplatin); PFL (cisplatin, 5-fluorouracil, leucovorin); IM (ifosfamide, mitomycin); IE (ifosfamide, etoposide); IP (ifosfamide, cisplatin); MIP (mitomycin, ifosfamide, cisplatin); ICE (ifosfamide, carboplatin, etoposide); PIE (cisplatin, ifosfamide, etoposide); Viorelbine and cisplatin; Carboplatin and paclitaxel; CAV (cyclophosphamide, doxorubicin, vincristine); CAE (cyclophosphamide, doxorubicin, etoposide); CAVE (cyclophosphamide, doxorubicin, vincristine, etoposide); EP (etoposide, cisplatin); and CMCcV (cyclophosphamide, methotrexate, lomustine, vincristine).

Patients identified as potentially responsive to Src inhibition therapy may also be treated using a Src pathway inhibitor in combination with systemic anti-cancer drugs, such as epithilones (BMS-247550, Epo-906), reformulations of taxanes (Abraxane, Xyotax), microtubulin inhibitors (MST-997, TTI-237), or with targeted cytotoxins such as CMD-193 and SGN-15. Additional useful anti-cancer agents include TAXOTERE®, TARCEVA®, GEMZAR® (gemcitabine), 5-FU, AVASTIN®, ERBITUX®, TROVAX®, anatumomab mafenatox, letrazole, docetaxel, and anthracyclines.

For combination therapies, a Src pathway inhibitor and additional therapeutic or diagnostic agents are administered within any time frame suitable for performance of the intended therapy or diagnosis. Thus, the single agents may be administered substantially simultaneously (i.e., as a single formulation or within minutes or hours) or consecutively in any order. For example, single agent treatments may be administered within about 1 year of each other, such as within about 10, 8, 6, 4, or 2 months, or within 4, 3, 2 or 1 week(s), or within about 5, 4, 3, 2 or 1 day(s).

### EXAMPLES

The invention is now described with reference to the following Examples. These Examples are provided for the purpose of illustration only, and the invention is not limited to these Examples, but rather encompasses all variations which are evident as a result of the teaching provided herein.

### Example 1

### Breast Cancer Tissue Microarrays

The HistoRx YTMA 49-7 breast cancer cohort contains 650 FFPE patient samples at 1× redundancy with a median follow-up time of 106 months.

Paraffin sections were deparaffinized in xylene and hydrated and then put in Tris EDTA buffer PT MODULE™ Buffer 4 (100×Tris EDTA Buffer, pH 9.0) TA-050-PM4X (Lab Vision Corporation of Fremont, Calif.) for antigen retrieval. Sections were then rinsed once in 1×TBS TWEEN® (Lab Vision of Fremont, Calif.) for 5 minutes and incubated in peroxidase block (Biocare Medical of Concord, Calif.) for 15 minutes followed by a rinse in 1×TBS TWEEN® for 5 minutes. Sections were blocked using Background Sniper (Biocare Medical of Concord, Calif.) for 15 minutes. Sections were incubated with the primary antibody cocktail: anti-biomarker antibody (species was either rabbit or mouse) and anti-pan-cytokeratin (where mouse anti-biomarker antibody was used, a rabbit anti-pan-cytokeratin was used; or visa versa) (Dako of Glostrup, Denmark, at a 1:50 concentration) diluted in DaVinci Green (Biocare Medical of Concord, Calif.) for 1 hours at room temperature. In this study rabbit anti-biomarker antibodies included: anti-phospho-Src at a dilution of 1:100 (Upstate, Millipore of Billerica, Mass., CAT # 7910); anti-FAK at a dilution of 1:250 (Cell Signaling Technology of Danvers, Mass., CAT # 3285); anti-Paxillin at a dilution of 1:250 (Labvision of Fremont, Calif., CAT # RB-10643-R7); anti-p130cas at a dilution of 1:300 (BD of Franklin Lakes, N.J., CAT # 610271); anti-ER at a dilution of 1:200 (Dako of Glostrup, Denmark, Clone 1D5); anti-PR at a dilution of 1:1000 (Dako, Clone PgR636-M3569); and anti-HER2 at a dilution of 1:1000 (Dako, polyclonal A0485).

Following three 5-minute rinses in 1×TBS Tween, slides were incubated in secondary antibody cocktail of goat anti-species EnVision labeled polymer HRP reagent (specificity for the species of the anti-biomarker antibody (DAKO, prepared per manufacturer's instructions) and goat anti-species Alexa Fluor 555 conjugate (with specificity for the species of the anti-pan-cytokeratin antibody utilized (Invitrogen A21429 diluted 1:200 into the EnVision) for 30 minutes in the dark, rinsed and then treated with Cy5 tyramide, diluted 1:50 in amplification buffer (Perkin Elmer SAT705A) for 10 minutes room temperature in the dark, mounted with Prolong anti-fade with DAPI (Invitrogen of Carlsbad, Calif.) and allowed to dry overnight. Each stained specimen was imaged using a PM-2000™ system (HistoRx of New Haven, Conn.) at 20× magnification. A board-certified pathologist reviewed an H&E stained serial section of the cohort to confirm tumor tissue presence in the samples. Images were evaluated for quality (staining quality, minimal pixel saturation, focus, minimum evaluable tissue present) prior to analysis. The biomarkers are quantified within cytoplasmic and nuclear compartments by AQUA® analysis to generate an AQUA® score of the relative biomarker concentration in the tissue sample (Camp et al., Nature Medicine, 2002, 8(11):1323-1327, and U.S. Pat. No. 7,219,016, which describes systems and methods for automatically quantifying and identifying the location of proteins or biomarkers within cell containing tissue samples, and which are hereby incorporated by reference in its entirety).

### Example 2

### Cluster Analysis of Phosphorylated Src (pSRc) in Tumors

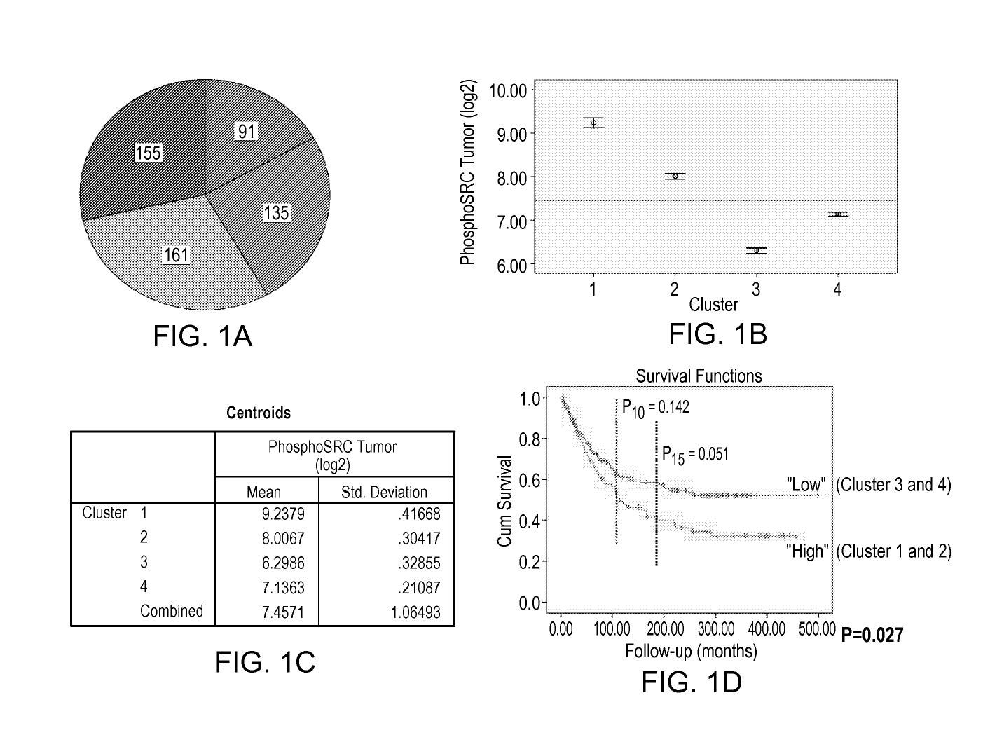

AQUA® score distribution frequency analysis and histograms were generated for biomarker expression in the tissue samples described in Example 1. Phosphorylated Src (pSrc) expression AQUA® scores obtained from analysis of the cohort ranged from 41.83 to 1458.19 with a median of 139.78 in tumor tissue. Two-step unsupervised cluster analysis of specific biomarker AQUA® scores obtained from the cohort analysis showed patients could be segregated into groups based on expression. Patients could be segregated into four groups based on phosphorylated Src expression: very low expression (Log transformed AQUA® score Mean 6.2986; 29% of patients); low expression (Log transformed AQUA® score Mean 7.1363; 29% of patients); intermediate expression (Log transformed AQUA® score Mean 8.0067; 25% of patients); and high expression (Log transformed AQUA® score Mean 9.2379; 17% of patients) (FIGS. 1A-1C). Kaplan Meier analysis of the combined two highest phosphorylated Src expressing groups versus the two lowest phosphorylated Src expressing groups showed an overall statistically significant survival advantage for the low expressing groups (p=0.027). (FIG. 1D).

### Example 3

### Cluster Analysis of p130cas Expression in Tumor and Tumor Cell Cytoplasm

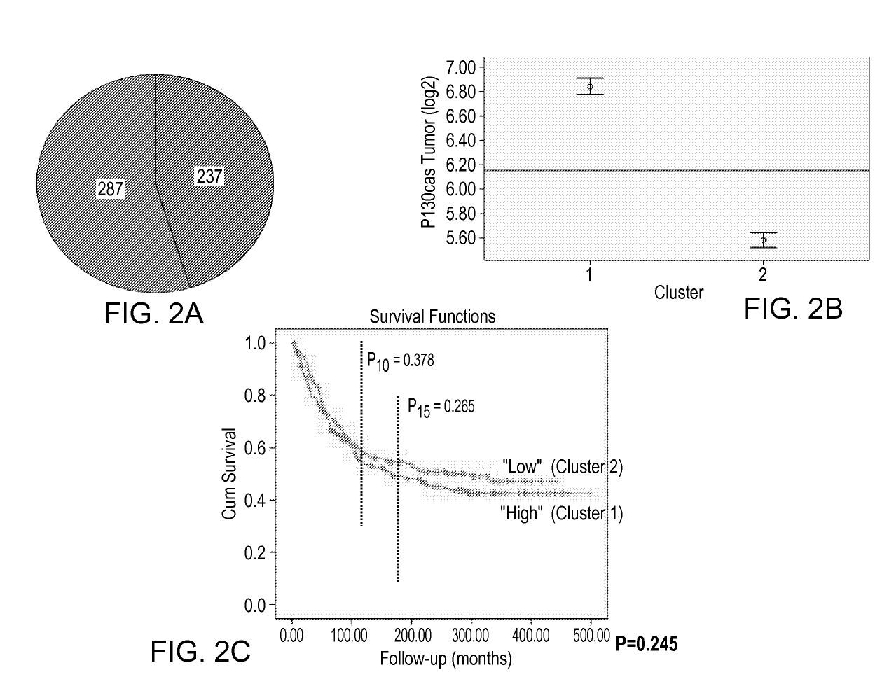

AQUA® score distribution frequency analysis and histograms were generated for biomarker expression in the tissue samples described in Example 1. The p130cas expression AQUA® scores were obtained from analysis of the cohort ranged from 12.12 to 327.10 with a median of 72.17 in tumor tissue. Two-step unsupervised cluster analysis of specific biomarker AQUA® scores obtained from the cohort analysis showed patients could be segregated into groups based on expression. Patients could be segregated into two groups based on p130cas expression: low expression (Log transformed AQUA® score Mean 5.5824; 55% of patients) and high expression (Log transformed AQUA® score Mean 6.8430; 45% of patients) (FIGS. 2A-2B). Kaplan Meier analysis of the low p130cas expressing group versus the high p130cas expressing group showed a slight survival advantage for the low expressing group; however it was not statistically significant (FIG. 2C).

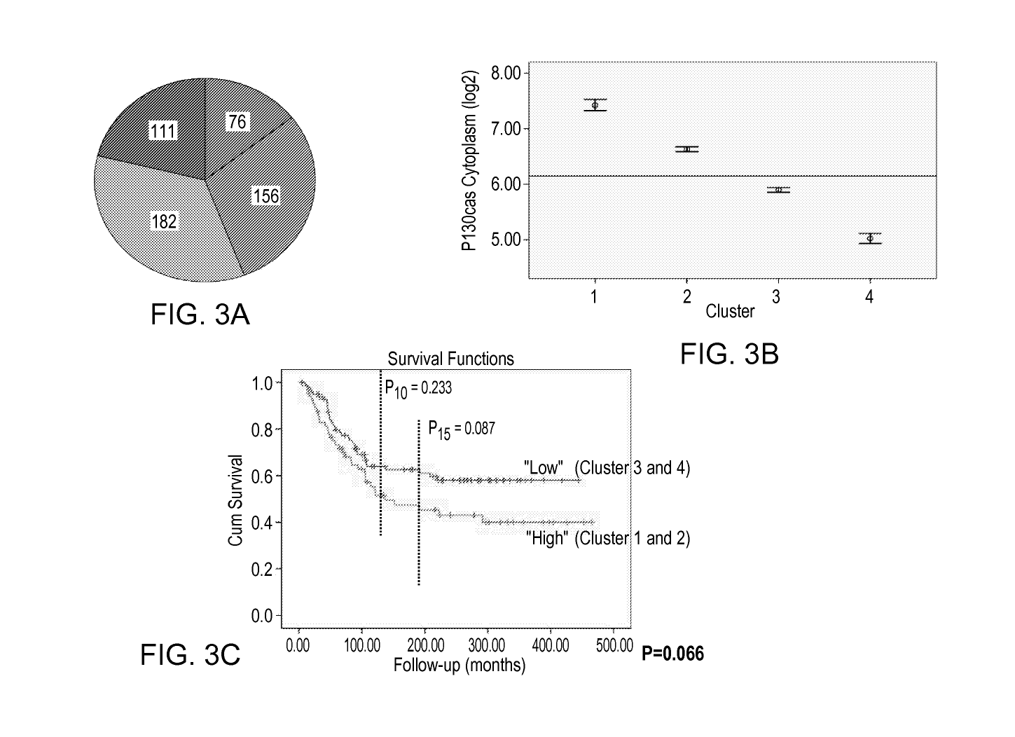

However, cluster analysis of patients based on AQUA® scores for tumor cell cytoplasmic expression of p130cas showed patients could be segregated into four groups based on p130cas expression: very low expression (Log transformed AQUA® score Mean 5.0264; 21% of patients); low expression (Log transformed AQUA® score Mean 5.8958; 35% of patients); intermediate expression (Log transformed AQUA® score Mean 6.6326; 30% of patients); and high expression (Log transformed AQUA® score Mean 7.4249; 14% of patients) (FIGS. 3A-3B). Kaplan Meier analysis of the combined two highest p130cas expressing groups versus the two lowest p130cas expressing groups showed a statistically significant survival advantage for the low expressing groups p=0.066 (for alpha=0.10) (FIG. 3C).

### Example 4

### Cluster Analysis of Paxillin Expression in Tumors

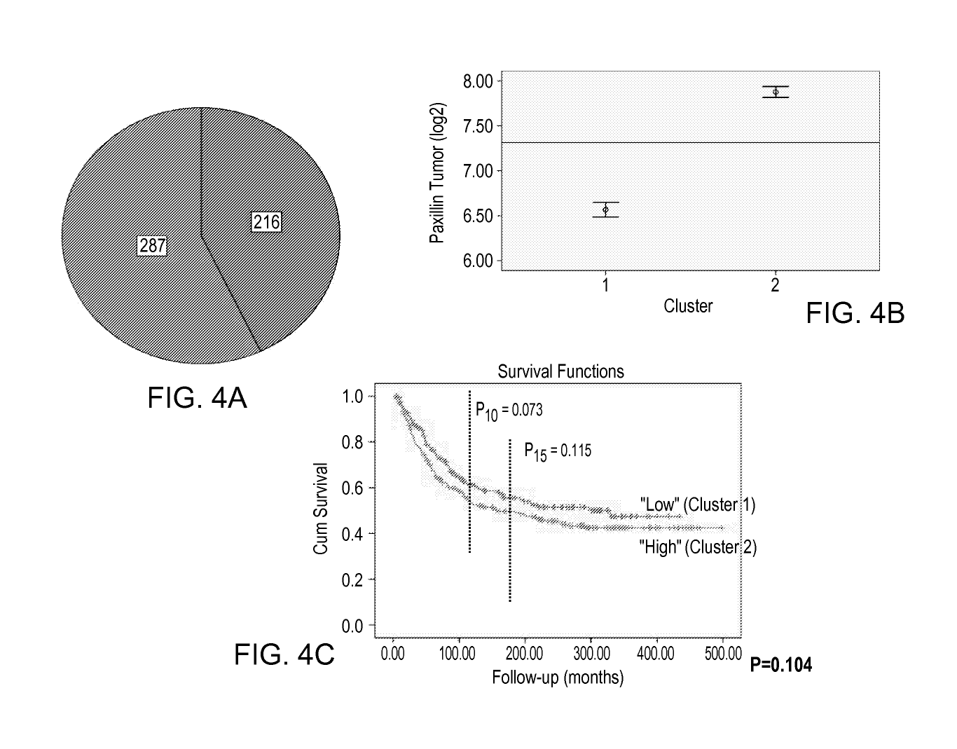

AQUA® score distribution frequency analysis and histograms were generated for biomarker expression in the tissue samples described in Example 1. Paxillin expression AQUA® scores obtained from analysis of the cohort ranged from 13.43 to 740.15 with a median of 171.58 in tumor tissue. Two-step unsupervised cluster analysis of specific biomarker AQUA® scores obtained from the cohort analysis showed patients could be segregated into groups based on expression. Patients could be segregated into two groups based on AQUA® scores for paxillin expression: low expression (Log transformed AQUA® score Mean 6.5671; 43% of patients) and high expression (Log transformed AQUA® score Mean 7.8770; 57% of patients) (FIGS. 4A-4B). Kaplan Meier analysis of the low expressing group versus the high expressing group showed a slight survival advantage for the low expressing group however it was not statistically significant (FIG. 4C).

### Example 5

### Cluster Analysis of FAK Expression in Tumors, Tumor Cell Nuclei and Tumor Cytoplasm

AQUA® score distribution frequency analysis and histograms were generated for biomarker expression in the tissue samples described in Example 1. FAK expression Log transformed AQUA® scores obtained from analysis of the cohort ranged from 5.37 to 51.22 with a median of 11.6 in tumor tissue. Two-step unsupervised cluster analysis of specific biomarker AQUA® scores obtained from the cohort analysis showed patients could be segregated into groups based on expression. Patients could be segregated into five groups based on AQUA® scores for FAK expression: very low expression (Log transformed AQUA® score Mean 2.8990; 18% of patients); low expression (Log transformed AQUA® score Mean 3.3422; 31% of patients); intermediate expression (Log transformed AQUA® score Mean 3.6892; 24% of patients); high expression (Log transformed AQUA® score Mean 4.0861; 19% of patients); and very high expression (Log transformed AQUA® score Mean 4.7737; 8% of patients). Kaplan Meier analysis of the groups showed no significant difference in patient outcome between these groupings.

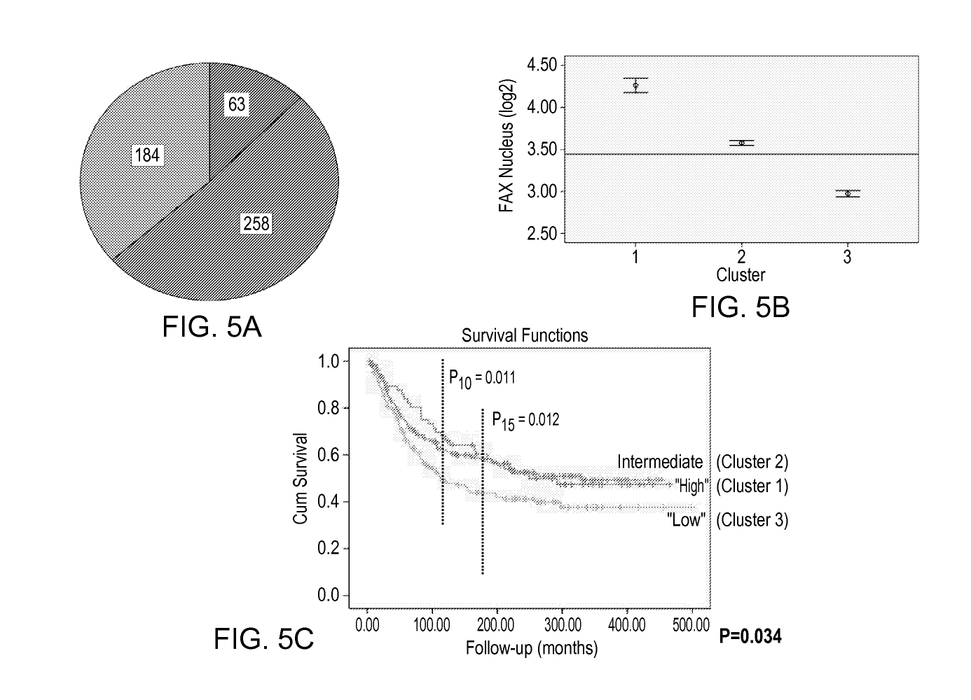

Cluster analysis of AQUA® scores for FAK expression in tumor cell nuclei segregated patients into three groups: low expression (Log transformed AQUA® score Mean 2.9728; 37% of patients); intermediate expression (Log transformed AQUA® score Mean 3.5773; 51% of patients); and high expression (Log transformed AQUA® score Mean 4.2619; 12% of patients) (FIGS. 5A-5B). Kaplan Meier analysis of the three groups showed a statistically significant survival advantage for the high and intermediate expression groups versus the low expressing group (p=0.034) (FIG. 5C).

Cluster analysis of AQUA® scores for FAK expression in tumor cell cytoplasm segregated patients into five groups: very low expression (Log transformed AQUA® score Mean 2.9426, 23% of patients); low expression (log transformed AQUA® score Mean 3.6059, 31% of patients); intermediate expression (Log transformed AQUA® score Mean 3.7791; 25% of patients); high expression (Log transformed AQUA® score Mean 4.2058; 14% of patients); and very high expression (Log transformed AQUA® score Mean 4.8737; 7% of patients). Kaplan Meier analysis did not show a significant survival advantage for any particular expression group for this particular data set.

### Example 6

### Cluster Analysis of HER2 Expression in Tumors

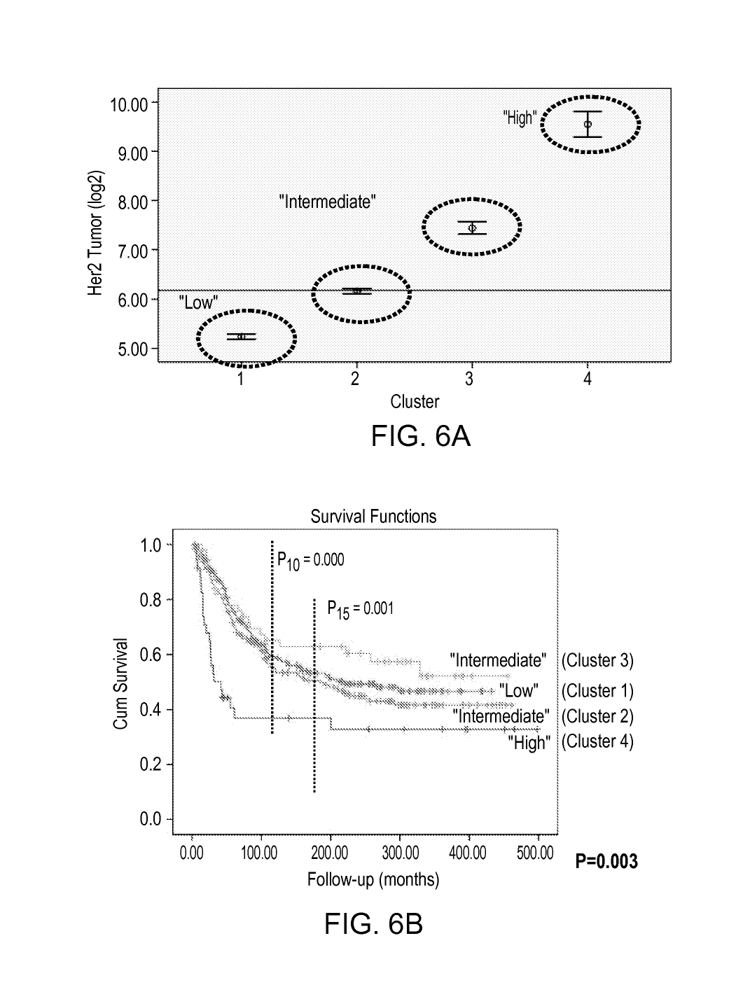

AQUA® score distribution frequency analysis and histograms were generated for biomarker expression in the tissue samples described in Example 1. HER2 expression Log transformed AQUA® scores obtained from analysis of the cohort ranged from 18.91 to 1713.07 with a median of 62.81 in tumor tissue. Two-step unsupervised cluster analysis of specific biomarker AQUA® scores obtained from the cohort analysis showed patients could be segregated into groups based on expression. Patients could be segregated into four groups based on AQUA® scores for HER2 expression: very low expression (Log transformed AQUA® score Mean 5.2378; 41% of patients); low expression (Log transformed AQUA® score Mean 6.1634; 40% of patients); intermediate expression (Log transformed AQUA® score Mean 7.4388; 11% of patients); and high expression (Log transformed AQUA® score Mean 9.5491; 8% of patients) (FIG. 6A). The two low groups were combined for Kaplan Meier analysis which showed the three groups had statistically significant survival differences for the high, intermediate and low expression groups (p=0.003) (FIG. 6B).

### Example 7

### Cluster Analysis of ER Expression in Tumor Cell Nuclei

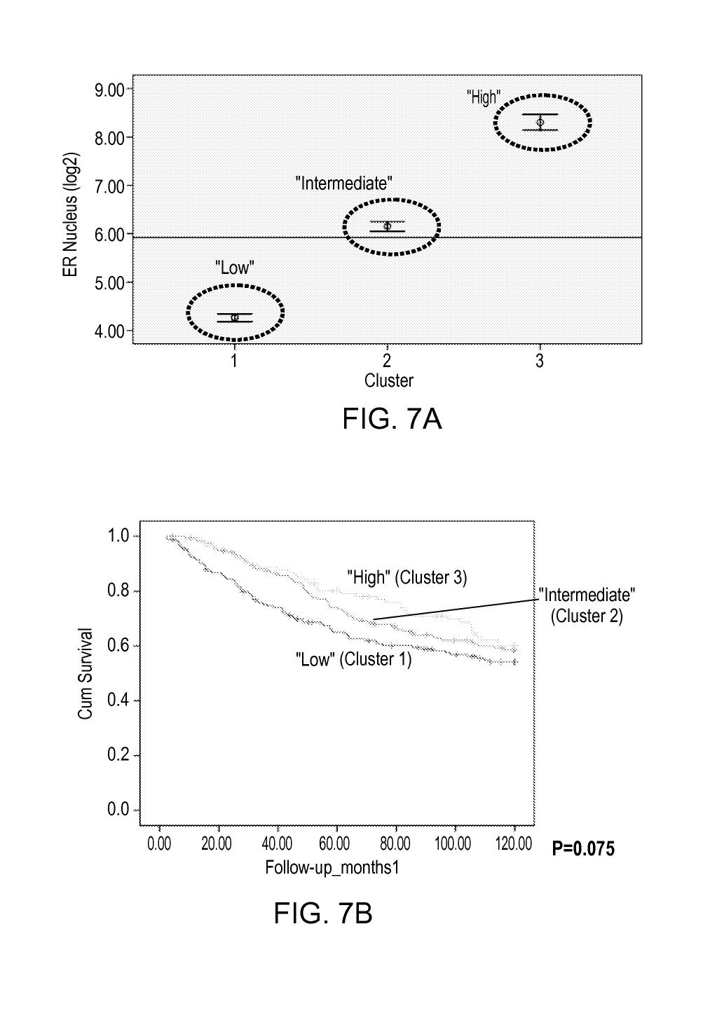

AQUA® score distribution frequency analysis and histograms were generated for biomarker expression in the tissue samples of the cohort. ER expression Log transformed AQUA® scores obtained from analysis of the cohort ranged from 10.69 to 1314.44 with a median of 51.95 in tumor cell nuclei. Two-step unsupervised cluster analysis of specific biomarker AQUA® scores obtained from the cohort analysis showed patients could be segregated into groups based on expression. Patients could be segregated into three groups based on AQUA® scores for ER expression: low expression (Log transformed AQUA® score Mean 4.1530; 46% of patients); intermediate expression (Log transformed AQUA® score Mean 5.3674; 35% of patients); and high expression (Log transformed AQUA® score Mean 7.0921; 19% of patients) (FIG. 7A). Kaplan Meier analysis looking at 10 year survival showed statistically significant survival differences for the high and low expression groups (p<0.075; FIG. 7B).

### Example 8

### Cluster Analysis of PR Expression in Tumor Cell Nuclei

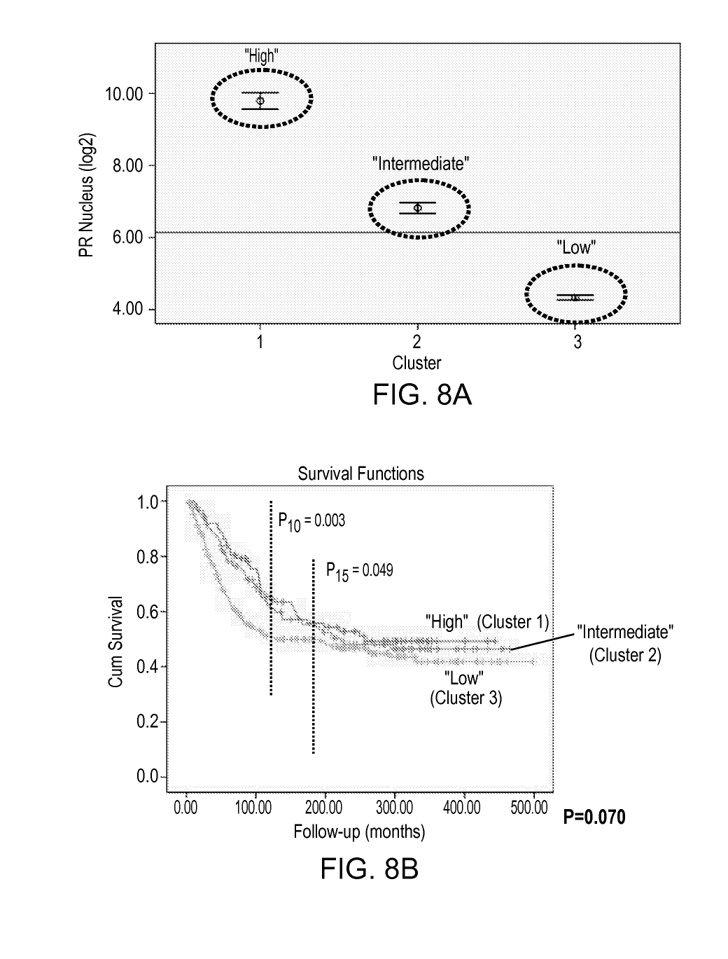

AQUA® score distribution frequency analysis and histograms were generated for biomarker expression in the tissue samples of the cohort. PR expression AQUA® scores obtained from analysis of the cohort ranged from 0 to 3134.23 with a median of 43.7. Two-step unsupervised cluster analysis of specific biomarker AQUA® scores obtained from the cohort analysis showed patients could be segregated into groups based on expression. Patients could be segregated into three groups based on AQUA® scores for PR expression: low expression (Log transformed AQUA® score Mean 4.4582; 59% of patients); intermediate expression (Log transformed AQUA® score Mean 6.3467; 28% of patients); and high expression (Log transformed AQUA® score Mean 8.9657; 13% of patients) (FIG. 8A). Kaplan Meier analysis showed a statistically significant survival advantage for the high expressing group (p₁₀=0.003) (FIG. 8B).

### Example 9

### Profiling of Breast Cancer Markers

Correlations between Src pathway markers and common breast cancer markers HER2, ER, and PR were investigated. To elucidate correlations between the biomarkers studied, a heat map chart was constructed in which the expression of each marker in each sample is indicated by color scale from low expression (green) to high expression (red). Samples were sorted based on HER2 expression. Results show that Src pathway marker expression including cytoplasmic FAK protein, p130cas, phosphorylated Src (pSrc), and paxillin expression are correlated and indicative of Src pathway activation. It was found that Src pathway markers are generally positively correlated with HER2 expression and negatively correlated with ER and PR expression. Spearman Rho correlations and their associated p values of significance are presented in Tables 1 and 2, respectively. These data demonstrate the strong positive correlation between Src pathway markers and the Her2 marker, as well as negative correlations with ER and PR.

Correlations between Src pathway markers and HER2, ER and PR were confirmed statistically by Spearman Rho correlations in which rank order correlations of the AQUA® scores for each biomarker was evaluated. As shown in Tables 1 and 2 (C=cytoplasm, N=nucleus), below, Src pathway markers, p130cas, paxillin, and cytoplasmic FAK protein are strongly correlated with phosphorylated Src. ER and PR were statistically significantly correlated with each other. Also Src pathway markers, particularly phosphorylated Src, p130cas, and paxillin, are strongly correlated with HER2.

TABLE 1

| Rho | | | | | | | |
|---|---|---|---|---|---|---|---|
| values* | HER2 C | ER N | PR N | pSrc C | P130cas C | Paxillin C | FAK C |
| HER2 C | | 0.006 | 0.049 | 0.4 | 0.465 | 0.431 | 0.283 |
| ER N | 0.006 | | 0.428 | 0.066 | 0.097 | 0.042 | 0.196 |
| PR N | 0.049 | 0.428 | | −0.005 | −0.013 | −0.029 | 0.093 |
| pSrc C | 0.4 | 0.066 | −0.005 | | 0.516 | 0.553 | 0.466 |
| P130cas C | 0.465 | 0.097 | −0.013 | 0.516 | | 0.725 | 0.479 |
| Paxillin C | 0.431 | 0.042 | −0.029 | 0.553 | 0.725 | | 0.321 |
| FAK C | 0.283 | 0.196 | 0.093 | 0.466 | 0.479 | 0.321 | |
| Rho | <0.2 | 0.2-0.4 | 0.4-0.6 | >0.6 | | | |

C, cytoplasmic and non-nuclear expression
N, nuclear expression
*Rho < 0.2 = weak correlation; 0.2 < Rho < 0.4 = moderate correlation; 0.4 < Rho < 0.6 = strong correlation; Rho > 0.6 = strong correlation

TABLE 2

| Rho | | | | | | | |
|---|---|---|---|---|---|---|---|
| values* | HER2 C | ER N | PR N | pSrc C | P130cas C | Paxillin C | FAK C |
| HER2 C | | 0.8935 | 0.3044 | <0.0001 | <0.0001 | <0.0001 | <0.0001 |
| ER N | 0.8935 | | <0.0001 | 0.1667 | 0.0414 | 0.382 | <0.0001 |
| PR N | 0.3044 | <0.0001 | | 0.92 | 0.7906 | 0.5432 | 0.0507 |
| pSrc C | <0.0001 | 0.1667 | 0.92 | | <0.0001 | <0.0001 | <0.0001 |
| P130cas C | <0.0001 | 0.0414 | 0.7906 | <0.0001 | | <0.0001 | <0.0001 |
| Paxillin C | <0.0001 | 0.382 | 0.5432 | <0.0001 | <0.0001 | | <0.0001 |
| FAK C | <0.0001 | <0.0001 | 0.0507 | <0.0001 | <0.0001 | <0.0001 | |

C, cytoplasmic and non-nuclear expression
N, nuclear expression
p values for correlations presented in Table 1 are indicated in this table (p < 0.05 considered significant).

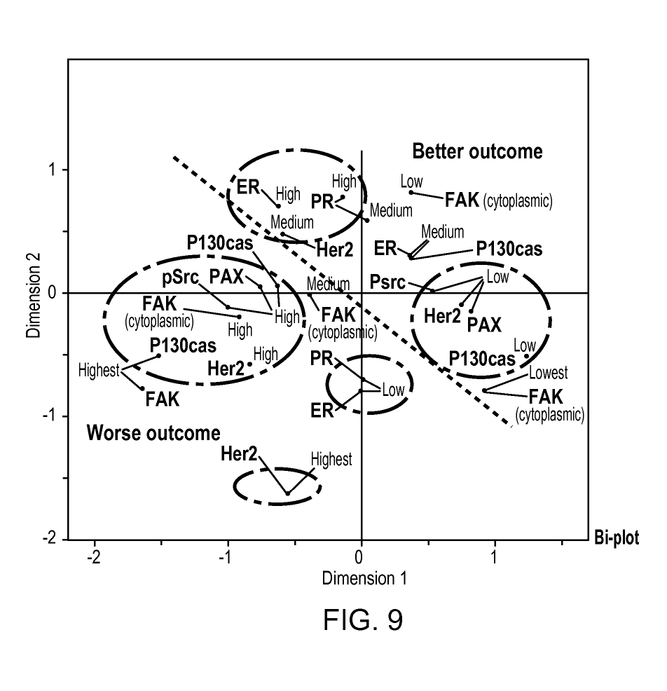

Multiple correspondence analysis (MCA) was used to further investigate associations amongst all biomarkers studied. This method uses categorical data, therefore patients were separated into groups based on the unsupervised cluster analysis of AQUA® scores for each biomarker. A biplot was created to provide a visual tool to identify relationships and associations amongst the groups. Groups characterized by marker expression that are closer together in two dimensional space on the biplot are associated (FIG. 9).

### Example 10

### Patient Segment Profiling

Patient groups were created based on expression of HER2, ER, PR and correlations within these groups with Src pathway marker expression were investigated. Patient groups were defined as shown below. The mean AQUA® scores for each marker for each of these groups are shown in Table 3 (C=cytoplasm, N=nucleus).

A1, High ER expression

A2, High PR expression

A3, High ER and PR expression

B, High HER2 expression

C, Low HER2 expression

D1, Low ER expression

D2, Low PR expression

D3, Low ER and Low PR expression

E, Low ER, PR and HER2 expression

TABLE 3

| | Age at | | | | | | | | |
|---|---|---|---|---|---|---|---|---|---|
| | Diagnosis | ER N | HER2 C | PR N | FAK N | FAK C | P130cas C | Paxillin C | pSRC C |
| A1 | Mean | Mean | Mean | Mean | Mean | Mean | Mean | Mean | Mean |
| A2 | 57.73 | 338.68 | 108.42 | 114.04 | 12.97 | 15.00 | 124.24 | 266.80 | 259.88 |
| A3 | 60.60 | 80.40 | 112.28 | 1262.80 | 11.02 | 13.32 | 80.43 | 209.17 | 213.71 |
| B | 66.33 | 391.22 | 108.20 | 1402.64 | 14.10 | 17.36 | 121.46 | 258.00 | 245.70 |
| C | 48.60 | 62.49 | 871.91 | 72.25 | 13.52 | 16.48 | 101.71 | 188.37 | 274.46 |
| D1 | 58.48 | 169.90 | 43.39 | 496.30 | 10.56 | 11.45 | 68.09 | 156.24 | 144.85 |
| D2 | 49.94 | 24.10 | 108.33 | 268.60 | 10.55 | 11.17 | 65.87 | 180.03 | 168.22 |
| D3 | 61.66 | 184.29 | 110.69 | 22.41 | 11.47 | 13.19 | 75.71 | 180.89 | 188.57 |
| E | 54.29 | 19.50 | 383.21 | 20.27 | 11.51 | 14.73 | 105.25 | 226.13 | 242.00 |
| A1 | 54.22 | 18.38 | 41.33 | 20.40 | 10.90 | 11.60 | 67.47 | 164.63 | 155.80 |

C, cytoplasmic expression
N, nuclear expression

Multivariate analysis was conducted based on three sets of data: 1) patient assignments to analytical groups based on cluster analysis of HER2, ER, PR expression ranging from lowest to highest; 2) cluster analysis of Src pathway markers categorizing patients into groups ranging from lowest to highest expression; 3) survival in terms of months from tumor removal to most recent event (death or censoring) and death. A model was developed using a backward stepwise selection criteria (WALD) in which the first step entered all valid cases and variables in the analysis. Subsequent steps tested the contribution of each variable against the baseline model. Those variables not contributing significantly to improvement in the baseline model were eliminated from subsequent steps. Backward stepping continued until no further improvement in the baseline model was observed or all variables were eliminated from the model. Resultant variables were deemed statistically significantly describing survival for the population under study. Hazard values for baseline are defined as equal to 1.000. Hazard values>1.000 are related to poorer survival. Hazard values<1.000 are related to better survival. By Cox analysis, only two patient groupings (both containing HER2) showed an improvement in survival prognostic by using additional markers (Table 4).

TABLE 4

| | | | | Cox Chi- | Hazard |
|---|---|---|---|---|---|
| Population | | | Time | Square | Ratio |
| of Interest | Marker | Expression | Period | (p =) | (Cox) |
| HER2 | FAK | Low | 10 years | 0.003 | 0.270 |
| | (cytoplasm) | | | | |
| | Paxillin | High | 10 years | 0.027 | 1.622 |
| ER-PR- | P130cas | High | 10 years | 0.039 | 12.500 |
| HER2 | Paxillin | Low | 10 years | 0.012 | 0.139 |

In the patients grouped by HER2 expression, higher expression of paxillin is associated with high expression of HER2 and worse survival and a significant increase in the Hazard ratio is seen when compared to prognosis based on HER2 alone. Lower cytoplasmic expression of FAK protein was associated with low HER2 and better survival characteristics, and a modest but significant change in the Hazard ratio was obtained in comparison to prognosis based on HER2 alone. In the patients grouped based on ER, PR, and HER2 expression, low paxillin expression was associated with low triple markers and better survival, and a modest but significant decrease in the Hazard ratio was observed as compared to prognosis based on ER, PR, and HER2 expression alone. High expression of p130cas was associated with high triple marker expression (ER, PR, HER2) and worse survival and a strong and significant increase in the Hazard ratio was observed in comparison to prognosis based just on ER, PR, and HER2 expression.

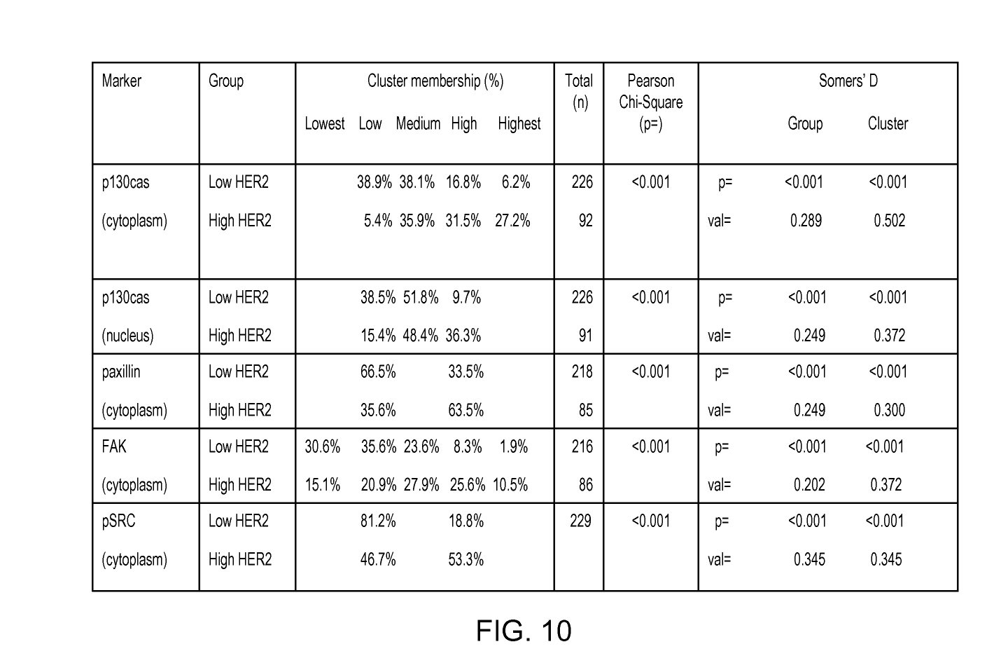

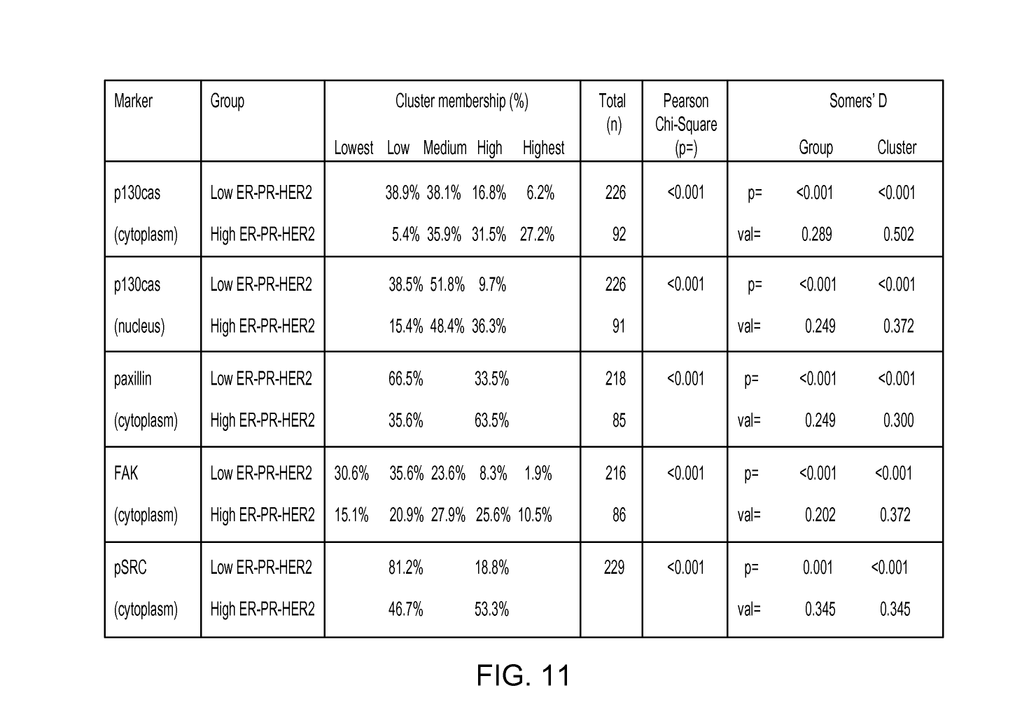

Biomarkers of Src activation and HER2 expression were correlated between two ordinal scales using Somers' D value for pairwise comparisons, the results of which are presented in FIG. 10. Somers' D values were also used to correlate biomarkers of Src activation and patient groups defined by expression of HER2, estrogen receptor, and progesterone receptor, the results of which are presented in FIG. 11. This analysis revealed an association between Src activation and expression of HER2, estrogen receptor, and progesterone receptor (HER2/ER/PR), with strong associations between HER2/ER/PR and paxillin expression, p130cas expression, and Src phosphorylation.

### Example 11

### Correlation of Src Pathway Markers In ER/PR Positive, HER2 Negative Patients

As shown in Table 1, Src pathway markers p130cas, paxillin, and cytoplasmic FAK protein are strongly correlated with phosphorylated Src (pSrc). Using Spearman's Rho analysis, statistically significant correlations were also observed between pSrc, p130cas, FAK and paxillin in a sub-population of ER and/or PR positive, HER2 negative patients as shown in Table 5. A double asterisk (**) indicates that the correlation is significant at the 0.01 level (two-tailed).

TABLE 5

| Spearman's rho | | pSRC_LOG2 | Paxillin_LOG2 | P130CAS_LOG2 | FAK_LOG2 |
|---|---|---|---|---|---|
| pSRC_LOG2 | Correlation | 1.000 | .574** | .525** | .564** |
| | Coefficient | | | | |
| | Sig. (2- | . | .000 | .000 | .000 |
| | tailed) | | | | |
| | N | 317 | 317 | 309 | 296 |
| Paxillin_LOG2 | Correlation | .574** | 1.000 | .730** | .393** |
| | Coefficient | | | | |
| | Sig. (2- | .000 | . | .000 | .000 |
| | tailed) | | | | |
| | N | 296 | 300 | 293 | 286 |
| P130CAS_LOG2 | Correlation | .525** | .730** | 1.000 | .544** |
| | Coefficient | | | | |
| | Sig. (2- | .000 | .000 | . | .000 |
| | tailed) | | | | |
| | N | 309 | 293 | 313 | 294 |
| FAK_LOG2 | Correlation | .564** | .393** | .544** | 1.000 |
| | Coefficient | | | | |
| | Sig. (2- | .000 | .000 | .000 | . |
| | tailed) | | | | |
| | N | 296 | 286 | 294 | 300 |

### Example 12

### Correlation of Individual Src Pathway Markers with Src Pathway Activation Status

As described in Example 2, expression levels of phosphorylated Src (pSrc) are a reliable measure of a patient's prognosis; lower levels of pSrc are associated with a statistically significant survival advantage. Levels of pSrc are also an indicator of the level of Src pathway activation (i.e., Src signaling), and are thus useful in assessing how well a patient will respond to therapy with a Src pathway inhibitor. Because pSrc and other Src pathway proteins are expressed in a continuous range of values in vivo, algorithms were developed to correlate the continuum of expression levels of various Src pathway proteins with a binary variable representing the status of Src pathway activation (i.e., SRC-ON or SRC-OFF). If a Src pathway protein was expressed beyond a certain threshold level, then the Src pathway was deemed activated to an extent that a patient is expected to derive an enhanced benefit from one or more of the therapeutic regimens described herein (i.e., SRC-ON). Exemplary benefits include increased overall response rates (ORR) or survival endpoints, such as such as progression free survival (PFS), disease free survival (DFS) or overall survival (OS).

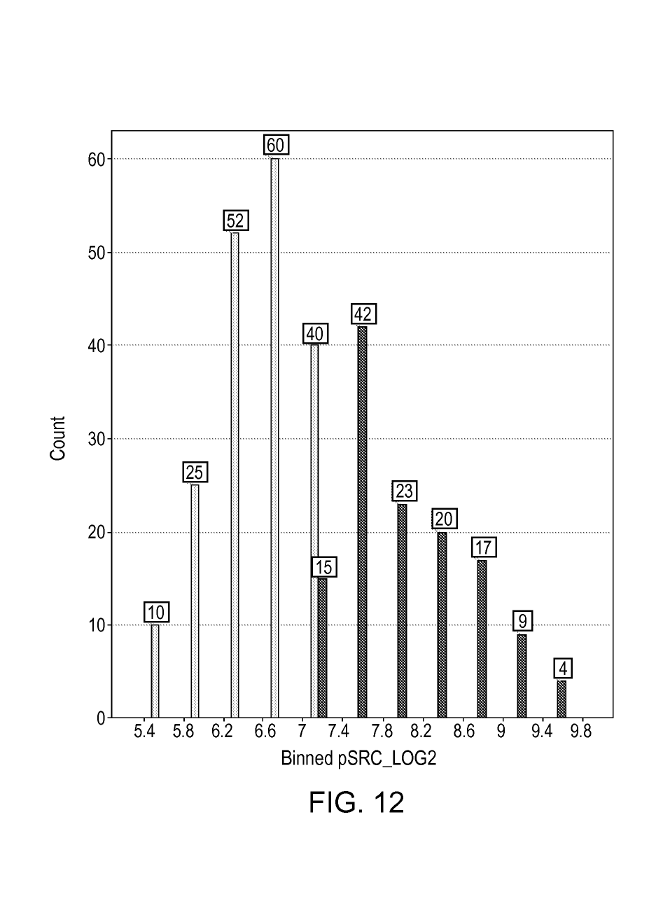

Using an unsupervised two-step clustering method (Zhang et al., 1996, Proceedings of the ACM SIGMOD Conference on Management of Data. Montreal, Canada: ACM), the same sub-population of ER and/or PR positive, HER2 negative patients described in Example 11 were clustered into two groups based on a natural “cut-point” observed in the pSrc expression AQUA® scores of the entire subset of patients, as shown in FIG. 12. Based upon this cut-point, 188 patients with pSrc AQUA® log2 transformed scores less than 7.28 were deemed SRC-OFF (i.e., the Src pathway is not activated to an extent in which treatment with a Src pathway inhibitor would be expected to provide an enhanced benefit, absent the consideration of other information to the contrary), while the remaining 129 patients with pSrc AQUA® log2 transformed scores greater than or equal to 7.28 were deemed SRC-ON. Accordingly, this latter group of patients would expect to derive an enhanced benefit from administration of a Src pathway inhibitor (absent the consideration of other information to the contrary).

However, due to the inherent difficulties in routinely determining phosphorylated protein levels in tissue specimens (Baker et al., Clin. Cancer Res., 2005, 11:4338-4340), the correlation of other Src pathway proteins as surrogate markers for pathway status were investigated. Expression levels of three other Src pathway markers (paxillin, p130cas, and FAK) were also found to independently indicate Src pathway status in the same sub-population of ER/PR positive, HER2 negative patients, as shown in Table 6. A univariate logistic regression model was used to determine the cut-points for each marker (coefficients estimated in the regression model), and their ability to univariately predict SRC-pathway status was examined.

TABLE 6

| Marker | | | | | | |
|---|---|---|---|---|---|---|
| (cut- | Regression | Standard | | % Overall | PPV | NPV |
| point) | Coefficient | Error | P-value | Agreement | (%) | (%) |
| Paxillin | 2.13 | 0.28 | <0.001 | 73.6 | 76.9 | 71.5 |
| (7.40) | | | | | | |
| p130 | 1.69 | 0.25 | <0.001 | 70.6 | 64.6 | 74.7 |
| (6.19) | | | | | | |
| FAK | 1.70 | 0.28 | <0.001 | 70.3 | 68.0 | 71.8 |
| (3.62) | | | | | | |

All three markers were entered into a multivariate model and no significant interactions were observed. Thus all three markers can be entered as a main effect as shown in Table 7.

TABLE 7

| Marker | Regression | Standard | |
|---|---|---|---|
| (cut-point) | Coefficient | Error | P-value |
| Paxillin (7.40) | 1.57 | 0.35 | <0.001 |
| p130 (6.19) | 0.67 | 0.25 | 0.05 |
| FAK (3.62) | 1.42 | 0.31 | <0.001 |

Based on this model, patients with paxillin AQUA® scores greater than or equal to 7.40, p130 AQUA® scores greater than or equal to 6.19, or FAK AQUA® scores greater than or equal to 3.62 would expect to derive an enhanced benefit from administration of a Src pathway inhibitor (absent the consideration of other information to the contrary).

### Example 13

### Correlation of Multiple Src Pathway Markers with Src Pathway Activation Status

In the interests of further maximizing sensitivity and specificity in identifying those patients that would expect to derive clinical benefit from the administration of a Src inhibitor (absent consideration of other information to the contrary), additional statistical approaches relying on the expression levels of multiple markers as surrogates for Src pathway activation were developed.

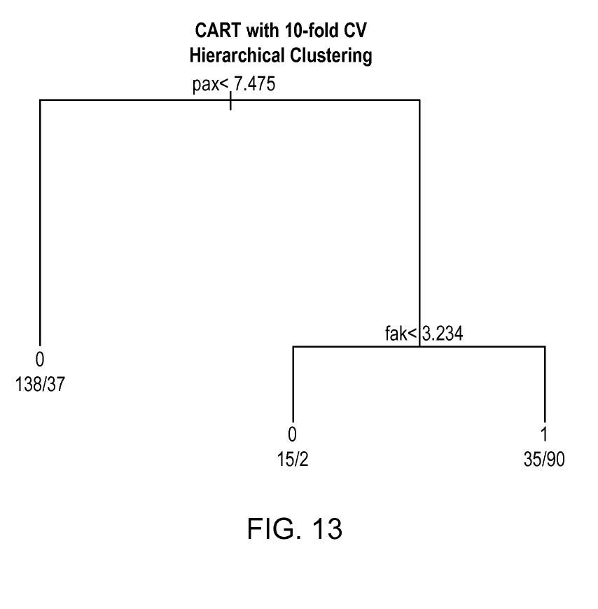

Data from the sub-population of ER and/or PR positive, HER2 negative patients described in Examples 11 and 12 were subjected to Classification and Regression Tree Modeling (CART) (Muller R., Mockel M., Clin. Chim. Acta, 2008, 394:1-6). In the first approach, pSrc log2 transformed AQUA® scores from these patients were used as definitive indicators of Src pathway activation status (i.e., patients were deemed SRC-ON when pSrc≧7.28 and SRC-OFF when pSrc<7.28). The CART model determined that the Src pathway was activated (i.e., SRC-ON) when the log2 transformed AQUA® scores of paxillin 7.475 and FAK 3.234, as shown in FIG. 13. This model demonstrated a positive predictive value (PPV) of 72% and a negative predictive value (NPV) of 79.7%. The CART model also determined that the Src pathway was not activated (i.e., SRC-OFF) when the log2 transformed AQUA® scores of paxillin<7.475 or FAK<3.234. This model correctly classified 243 out of 317 patients (76.7%), as shown in Table 8.

TABLE 8

| | | pSRC- | | |
|---|---|---|---|---|
| | | Designation (Reference) | | |
| | | On | Off | Total |
| CART | On | 90 | 35 | 125 |
| Designation | Off | 39 | 153 | 192 |
| Total | | 129 | 188 | 317 |

Overall % agreement: 76.7% (95% CI: 71.8-80.9)
Positive % agreement: 72.0% 95% CI: 65.8-77.4)
Negative % agreement: 79.7% 95% CI: 75.6-83.2)

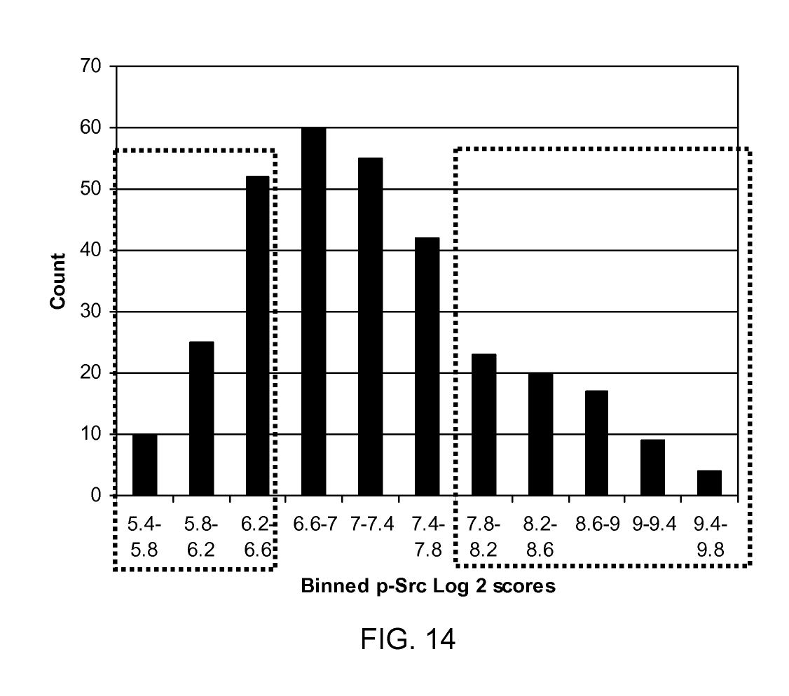

In a different approach, data from the sub-population of ER and/or PR positive, HER2 negative patients described in Examples 11 and 12 were subjected to CART in which pSrc AQUA® scores were divided into quartiles, rather than two distribution groups. In this particular model cut-points for SRC-OFF and SRC-ON were established by the upper end-point of the lowest quartile and the lower-end point of the highest quartile respectively. The middle two quartiles were designated intermediate (Int), as shown in FIG. 14. The designation of upper and lower quartiles provided two separate cut-points in pSrc data, and provided the basis for two separate CART analyses for evaluating the value of paxillin, p130 and FAK AQUA® scores as surrogate markers for Src pathway activation.

Using the upper quartile of pSrc expression as the cut-point, patients in the highest quartile of pSrc AQUA® scores were deemed SRC-ON and the patients in the remaining three quartiles SRC-OFF. Accordingly, patients in which the Src signaling pathway is activated (i.e., SRC-ON) in this model have a higher average level of expression of pSrc than the SRC-ON group from the previous model. In this model, Src pathway activation (i.e., SRC-ON) was predicted when the log2 transformed AQUA® scores of FAK 3.318 and paxcillin 7.704. The positive predictive value (PPV) of this method was 55.68% and the negative predictive value (NPV) was 86.40%.

Using the lower quartile of pSrc expression as the cut-point, patients in the lowest quartile of pSrc AQUA® scores were defined as SRC-OFF and the patients in the remaining three quartiles SRC-ON. Accordingly, patients in which the Src signaling pathway is activated (i.e., SRC-ON) in this model require only a very minimal level of expression of pSrc than the SRC-ON group from either of the two previous models. A lack of significant Src pathway activation (i.e., SRC-OFF) was predicted when the log2 transformed AQUA® scores of FAK<3.318 and p130<6.495. The positive predictive value (PPV) of this method was 90.13% and the negative predictive value (NPV) was 61.29%.

In each of these approaches, patients defined as SRC-ON would be expected to derive clinical benefit, or an enhanced clinical benefit, from a Src inhibitor; conversely, patients in the SRC-OFF group would not be expected to derive significant clinical benefit from such a therapeutic intervention, whether given alone as a monotherapy or in combination with other anticancer drugs.

### Example 14

### Src Pathway Activation Status In Triple Negative Breast Cancer Tumors

It was also discovered that Src pathway activation status (i.e., Src signaling) in HER2-negative, ER-negative, PR-negative (triple negative) breast cancer tumors could be determined by correlation with levels of Src pathway marker expression. Approximately 50% of patients with these tumors have high Src pathway marker expression and as previously described in the examples, Src pathway activation is predictive of a worse prognosis. As triple negative breast cancer patients have a particularly poor prognosis and few treatment options, the ability to identify those with an active Src pathway allows for the identification of patients most likely to respond to therapy with a Src inhibitor and the opportunity to improve patient outcome.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A method of predicting poor prognosis of a cancer patient comprising observing or detecting activation of Src signaling in a tumor sample obtained from the patient to thereby determine poor prognosis of the patient.

2. The method of claim 1, wherein the cancer is breast cancer.

3. The method of claim 1, wherein the patient has not received Src inhibition therapy.

4. The method of claim 1, wherein observing or detecting activation of Src signaling comprises observing or detecting expression level of a Src signaling marker.

5. The method of claim 4, wherein observing or detecting expression of the Src signaling marker comprises observing or detecting RNA or protein levels of the Src signaling marker.

6. The method of claim 5, wherein observing or detecting activation of Src signaling comprises observing or detecting elevated expression of p130cas.

7. The method of claim 5, wherein observing or detecting activation of Src signaling comprises observing or detecting elevated cytoplasmic expression of p130cas protein.

8. The method of claim 4, wherein observing or detecting activation of Src signaling comprises observing or detecting elevated expression of paxillin.

9. The method of claim 4, wherein observing or detecting activation of Src signaling comprises observing or detecting reduced nuclear expression of FAK.

10. The method of claim 4, wherein observing or detecting activation of Src signaling comprises observing or detecting elevated cytoplasmic expression of FAK protein.

11. The method of claim 1, wherein observing or detecting activation of Src signaling comprises observing or detecting an elevated level of a Src signaling protein in an activated state.

12. The method of claim 11, wherein observing or detecting activation of the Src signaling marker comprises observing or detecting an elevated level of phosphorylated Src tyrosine kinase.

13. The method of claim 1, wherein the tumor sample comprises high levels of HER2 expression.

14. The method of claim 1, wherein the tumor sample comprises low levels of estrogen receptor expression.

15. The method of claim 1, wherein the tumor sample comprises low levels of progesterone receptor expression.

16. The method of claim 13, wherein the tumor sample comprises low levels of estrogen receptor expression.

17. The method of claim 13, wherein the tumor sample comprises low levels of progesterone receptor expression.

18. The method of claim 16, wherein the tumor sample comprises low levels of progesterone receptor expression.

19. The method of claim 6, further comprising observing or detecting elevated expression of paxillin.

20. The method of claim 6, further comprising observing or detecting reduced nuclear expression of FAK.

21. The method of claim 6, further comprising observing or detecting elevated cytoplasmic expression of FAK protein.

22. The method of claim 6, further comprising observing or detecting an elevated level of phosphorylated Src tyrosine kinase.

23. The method of claim 19, further comprising observing or detecting reduced nuclear expression of FAK.

24. The method of claim 19, further comprising observing or detecting elevated cytoplasmic expression of FAK protein.

25. The method of claim 19, further comprising observing or detecting an elevated level of phosphorylated Src tyrosine kinase.

26. The method of claim 20, further comprising observing or detecting an elevated level of phosphorylated Src tyrosine kinase.

27. The method of claim 23, further comprising observing or detecting an elevated level of phosphorylated Src tyrosine kinase.

28. The method of claim 1, further comprising the step of observing or detecting elevated expression of HER2 in the tumor sample.

29. The method of claim 1, further comprising the step of observing or detecting reduced expression of estrogen receptor in the tumor sample.

30. The method of claim 1, further comprising the step of observing or detecting reduced expression of estrogen receptor in the tumor sample.

31. The method of claim 28, further comprising the step of observing or detecting reduced expression of estrogen receptor in the tumor sample.

32. The method of claim 28, further comprising the step of observing or detecting reduced expression of progesterone receptor in the tumor sample.

33. The method of claim 31, further comprising the step of observing or detecting reduced expression of progesterone receptor in the tumor sample.

34. The method of claim 29, further comprising the step of observing or detecting reduced expression of progesterone receptor in the tumor sample.

35. A method of predicting favorable prognosis of a cancer patient comprising observing or detecting suppression of Src signaling in a tumor sample obtained from the patient to thereby determine favorable prognosis of the patient.

36. The method of claim 35, wherein the cancer is breast cancer.

37. The method of claim 35, wherein the patient has not received Src inhibition therapy.

38. The method of claim 35, wherein observing or detecting suppression of Src signaling comprises observing or detecting expression level of a Src signaling marker.

39. The method of claim 38, wherein observing or detecting expression of the Src signaling marker comprises observing or detecting RNA or protein levels of the Src signaling marker.

40. The method of claim 39, wherein observing or detecting suppression of Src signaling comprises observing or detecting reduced expression of p130cas.

41. The method of claim 39, wherein observing or detecting suppression of Src signaling comprises observing or detecting reduced cytoplasmic expression of p130cas protein.

42. The method of claim 38, wherein observing or detecting suppression of Src signaling comprises observing or detecting reduced expression of paxillin.

43. The method of claim 38, wherein observing or detecting suppression of Src signaling comprises observing or detecting elevated nuclear expression of FAK protein.

44. The method of claim 38, wherein observing or detecting suppression of Src signaling comprises observing or detecting reduced cytoplasmic expression of FAK protein.

45. The method of claim 35, wherein observing or detecting suppression of Src signaling comprises observing or detecting a reduced level of a Src signaling protein in an activated state.

46. The method of claim 45, wherein observing or detecting suppression of the Src signaling marker comprises observing or detecting a reduced level of phosphorylated Src tyrosine kinase.

47. The method of claim 35, wherein the tumor sample comprises low levels of HER2 expression.

48. The method of claim 35, wherein the tumor sample comprises high levels of estrogen receptor expression.

49. The method of claim 35, wherein the tumor sample comprises high levels of progesterone receptor expression.

50. The method of claim 47, wherein the tumor sample comprises high levels of estrogen receptor expression.

51. The method of claim 47, wherein the tumor sample comprises high levels of progesterone receptor expression.

52. The method of claim 50, wherein the tumor sample comprises high levels of progesterone receptor expression.

53. The method of claim 40, further comprising observing or detecting reduced expression of paxillin.

54. The method of claim 40, further comprising observing or detecting elevated nuclear expression of FAK protein.

55. The method of claim 40, further comprising observing or detecting reduced cytoplasmic expression of FAK protein.

56. The method of claim 40, further comprising observing or detecting a reduced level of phosphorylated Src tyrosine kinase.

57. The method of claim 53, further comprising observing or detecting elevated nuclear expression of FAK protein.

58. The method of claim 53, further comprising observing or detecting reduced cytoplasmic expression of FAK protein.

59. The method of claim 53, further comprising observing or detecting a reduced level of phosphorylated Src tyrosine kinase.

60. The method of claim 54, further comprising observing or detecting a reduced level of phosphorylated Src tyrosine kinase.

61. The method of claim 57, further comprising observing or detecting a reduced level of phosphorylated Src tyrosine kinase.

62. The method of claim 35, further comprising the step of observing or detecting reduced expression of HER2 in the tumor sample.

63. The method of claim 35, further comprising the step of observing or detecting elevated expression of estrogen receptor in the tumor sample.

64. The method of claim 35, further comprising the step of observing or detecting elevated expression of estrogen receptor in the tumor sample.

65. The method of claim 62, further comprising the step of observing or detecting elevated expression of estrogen receptor in the tumor sample.

66. The method of claim 62, further comprising the step of observing or detecting elevated expression of progesterone receptor in the tumor sample.

67. The method of claim 65, further comprising the step of observing or detecting elevated expression of progesterone receptor in the tumor sample.

68. The method of claim 63, further comprising the step of observing or detecting elevated expression of progesterone receptor in the tumor sample.

69. A method of predicting favorable prognosis of a cancer patient comprising:
(a) observing or detecting reduced expression of HER2, estrogen receptor, and progesterone receptor in a tumor sample obtained from the patient; and
(b) observing or detecting reduced expression of paxillin in the tumor sample.

70. A method of predicting favorable prognosis of a cancer patient comprising:
(a) observing or detecting reduced expression of HER2, estrogen receptor, and progesterone receptor in a tumor sample obtained from the patient; and
(b) observing or detecting an elevated level of phosphorylated Src in the tumor sample.

71. A method of predicting poor prognosis of a cancer patient comprising:
(a) observing or detecting elevated expression of HER2, estrogen receptor, and progesterone receptor in a tumor sample obtained from the patient; and
(b) observing or detecting elevated expression of p130cas in the tumor sample.

72. A method of predicting favorable prognosis of a cancer patient comprising:
(a) observing or detecting reduced expression of HER2 in a tumor sample obtained from the patient; and
(b) observing or detecting reduced cytoplasmic expression of FAK protein or elevated nuclear expression of FAK protein in the tumor sample.

73. A method of predicting poor prognosis of a cancer patient comprising:
(a) observing or detecting elevated expression of HER2 in a tumor sample obtained from the patient; and
(b) observing or detecting elevated expression of paxillin in the tumor sample.

74. A method of performing an assay useful for predicting prognosis of a cancer patient comprising observing or detecting activation or suppression of Src signaling in a tumor sample obtained from the patient.

75. The method of claim 74, wherein observing or detecting a level of Src signaling comprises observing or detecting one or more of expression of p130cas, expression of paxillin, nuclear expression of FAK protein, cytoplasmic expression of FAK protein, and phosphorylation of Src tyrosine kinase.

76. The method of claim 74, wherein observing or detecting activation of Src signaling indicates a poor prognosis, and wherein observing or detecting suppression of Src signaling indicates a favorable prognosis.

77. A method of identifying cancer patients that may benefit from Src inhibition therapy comprising observing or detecting one or more of elevated expression of p130cas, elevated expression of paxillin, reduced nuclear expression of FAK protein, elevated cytoplasmic expression of FAK protein, and elevated phosphorylated Src tyrosine kinase, in a tumor sample obtained from the patient.

78. The method of claim 77, wherein observing or detecting elevated expression of p130cas comprises detecting elevated cytoplasmic p130cas protein.

79. The method of claim 77, wherein the cancer is breast cancer and the tumor sample:
(a) is HER2-positive;
(b) is estrogen receptor (ER)-positive;
(c) is progesterone receptor (PR)-positive;
(d) is estrogen receptor (ER)-positive and progesterone (PR)-receptor-positive; or
(e) is HER2-negative, estrogen receptor (ER)-negative, and progesterone (PR)-receptor negative.

80. The method of claim 77, wherein the cancer is breast cancer and further comprising:
(a) observing or detecting elevated HER2 expression in the tumor sample;
(b) observing or detecting elevated estrogen receptor expression in the tumor sample;
(c) observing or detecting elevated progesterone receptor expression in the tumor sample;
(d) observing or detecting elevated estrogen receptor expression and elevated progesterone receptor expression in the tumor sample; or
(e) observing or detecting reduced HER2 expression, reduced estrogen expression, and reduced progesterone expression in the tumor sample.

81. A method of identifying breast cancer patients that may benefit from Src inhibition therapy comprising observing or detecting activated Src signaling and detecting (a) elevated HER2 expression; (b) elevated estrogen receptor expression;
(c) elevated progesterone receptor expression; (d) elevated estrogen receptor expression and elevated progesterone receptor expression; or (e) reduced HER2 expression, reduced estrogen expression, and reduced progesterone expression; in the tumor sample.

82. A method of treating cancer in a patient comprising:
(a) observing or detecting one or more of elevated expression of p130cas, elevated expression of paxillin, reduced nuclear expression of FAK protein, elevated cytoplasmic expression of FAK protein, and elevated phosphorylated Src tyrosine kinase, in a tumor sample obtained from the patient; and
(b) administering an inhibitor of Src signaling to the patient.

83. The method of claim 82, wherein the cancer is breast cancer.

84. A method of treating cancer in a patient, wherein the cancer is characterized by one or more of elevated expression of p130cas, elevated expression of paxillin, reduced nuclear expression of FAK protein, elevated cytoplasmic expression of FAK protein, and elevated phosphorylated Src tyrosine kinase, comprising administering an inhibitor of Src signaling to the patient.

85. The method of claim 84, wherein the cancer is breast cancer.

86. A method of treating breast cancer in a patient comprising:
(a) observing or detecting activated Src signaling and (i) elevated HER2 expression; (ii) elevated estrogen receptor expression; (iii) elevated progesterone receptor expression; (iv) elevated estrogen receptor and elevated progesterone receptor expression; or (v) reduced HER2, reduced estrogen receptor, and reduced progesterone receptor expression; and
(b) administering an inhibitor of Src signaling to the patient.

87. A method of treating breast cancer in a patient, wherein the cancer is characterized by activated Src signaling and (a) elevated HER2 expression; (b) elevated estrogen receptor expression; (c) elevated progesterone receptor expression; (d) elevated estrogen receptor and elevated progesterone receptor expression; or (e) reduced HER2, reduced estrogen receptor, and reduced progesterone receptor expression; comprising administering an inhibitor of Src signaling to the patient.

88. A method of predicting the prognosis of a cancer patient comprising:
(a) obtaining a biological sample comprising a cancer cell from the cancer patient;
(b) subjecting the biological sample to protein or RNA expression analysis;
(c) quantifying the protein or RNA expression level of at least one Src pathway activation marker in the biological sample;
(d) calculating a score from the protein or RNA expression level of the at least one Src pathway activation marker in the biological sample; and
(e) using the score to predict the prognosis of the cancer patient.

89. A method of predicting the response of a cancer patient to a Src pathway inhibitor comprising:
(a) obtaining a biological sample comprising a cancer cell from the cancer patient;
(b) subjecting the biological sample to protein or RNA expression analysis;
(c) quantifying the protein or RNA expression level of at least one Src pathway activation marker in the biological sample;
(d) calculating a score from the protein or RNA expression level of the at least one Src pathway activation marker in the biological sample; and
(e) using the score to predict the response of the cancer patient to the Src pathway inhibitor.

90. A method of treating cancer in a cancer patient comprising:
(a) obtaining a biological sample comprising a cancer cell from the cancer patient;
(b) subjecting the biological sample to protein or RNA expression analysis;
(c) quantifying the protein or RNA expression level of at least one Src pathway activation marker in the biological sample;
(d) calculating a score from the protein or RNA expression level of the at least one Src pathway activation marker in the biological sample; and
(e) administering a Src pathway inhibitor to the cancer patient if the score is greater than or equal to at least one predetermined value.

91. The method of claim 90, wherein the cancer cell is ER and/or PR negative, HER2 positive.

92. The method of claim 91, wherein step (c) is carried out by AQUA® analysis.

93. The method of claim 92, wherein the score of step (d) and the predetermined value of step (e) is a log2 transformed AQUA® score.

94. The method of claim 93, wherein the at least one Src pathway activation marker is selected from the group consisting of phosphorylated Src (pSrc), p130cas, paxillin, and FAK.

95. The method of claim 93, wherein the at least one predetermined value is selected from the group consisting of:
(a) 7.28 if the Src pathway activation marker is phosphorylated Src;
(b) 6.19 if the Src pathway activation marker is p130cas;
(c) 7.40 if the Src pathway activation marker is paxillin; and
(d) 3.62 if the Src pathway activation marker is FAK.

96. The method of claim 93, wherein the at least one predetermined value is selected from the group consisting of:
(a) 7.704 if the Src pathway activation marker is paxillin; and
(b) 6.498 if the Src pathway activation marker is p130cas.

97. The method of claim 93, wherein the at least one Src pathway activation marker is paxillin and FAK.

98. The method of claim 97, wherein the at least one predetermined value is selected from the group consisting of:
(a) 7.475 for paxillin and 3.234 for FAK; and
(b) 7.704 for paxcillin and 3.318 for FAK.

99. The method of claim 97, wherein the at least one predetermined value is 6.495 for paxcillin and 3.318 for FAK.
